(19) United States
(12) Patent Application Publication
(10) Pub. No.: US 2018/0016351 A1
(43) Pub. Date: Jan. 18, 2018

(54) ANTIBODIES COMPRISING SEQUENCES FROM CAMELIDAE TO HIGHLY CONSERVED TARGETS

(71) Applicant: ARGEN-X N.V., Breda (NL)

(72) Inventors: Johannes Joseph Wilhelmus DE HAARD, Breda (NL); Christophe Frederic Jerome BLANCHETOT, Breda (NL)

(21) Appl. No.: 15/718,984

(22) Filed: Sep. 28, 2017

Related U.S. Application Data
(62) Division of application No. 14/415,370, filed on Jan. 16, 2015.

(30) Foreign Application Priority Data
Jul. 20, 2012 (GB) 1212940.9

(51) Int. Cl. C07K 16/42; C07K 16/00; B01D 15/18; G01N 33/68; C12N 15/10; B01D 15/38; C07K 16/28

(52) U.S. Cl. CPC C07K 16/4241; B01D 15/3809; C07K 16/00; C07K 16/2863; C07K 16/4208; G01N 33/686; C12N 15/1055; B01D 15/1864; C07K 16/4266; C07K 2317/22; C07K 2317/24; C07K 2317/31; C07K 2317/33; C07K 2317/14

(57) ABSTRACT

The present invention is concerned with antigen binding polypeptides, and in particular conventional antibodies derived from camelid species, that specifically bind to target antigens that are either self-antigens or highly conserved antigens. The present invention also relates to anti-idiotype antigen binding peptides which bind to a target antigen comprising the variable region of an antibody obtained from a species within the family Camelidae.

### FIELD OF THE INVENTION

The present invention is concerned with antigen binding polypeptides, and in particular conventional antibodies derived from camelid species, that specifically bind to target antigens that are either self-antigens or highly conserved antigens.

### BACKGROUND OF THE INVENTION

Monoclonal antibodies have many applications as research tools and, increasingly, as therapeutic or diagnostic agents. In particular, therapeutic antibody development is one of the fastest growing areas of the pharmaceutical industry. Generating high-quality monoclonal antibodies against a given therapeutic target is critical for success in the development of therapeutic antibody drugs. To date, a number of different technology platforms have been developed which allow production of monoclonal antibodies, including fully human or “humanized” antibodies, against target antigens of therapeutic interest. These technology platforms may be broadly divided into the in vivo approaches, based on use of immunised animals, and in vitro approaches, in which antibody sequences are selected from antibody variable chain cDNA libraries, using techniques such as phage display.

Notwithstanding the availability of both in vivo and in vitro platforms for monoclonal antibody development, particular difficulties still arise with the production of antibodies with binding specificity for antigens which are “highly conserved” across species and with production of antibodies against “self” antigens. Zhuo. et al., PLoS ONE, Vol. 4, Issue 6, June 2009 discuss the particular difficulties which arise in the use of conventional monoclonal antibody approaches based on immunization of animals (specifically mice) in the generation of antibodies against mouse self-antigens or antigens which are highly conserved between mice and humans, due to immune tolerance.

Previous attempts to overcome the immune tolerance associated with self or highly conserved antigens have included the use of specific knockout mice or the use of mouse strains with impaired immune tolerance. However, there are drawbacks associated with each of these approaches; generation of a knock-out mouse for each conserved antigen of interest is costly and time-consuming, whereas antibodies raised by immunisation of mice with impaired tolerance may be of variable quality, i.e. may display low affinity and/or low specificity for the target antigen.

Elsewhere it has been reported that human antibodies against human antigens can be isolated from non-immune human phage display libraries (Griffiths et al. EMBO J. Vol. 12., 725-734, 1993). The potential drawback of this approach is that synthetic phage display libraries only approximate the functional diversity naturally present in the human germline, thus the diversity is somewhat limited. Also, antibodies generated using non-immune libraries are not derived from in vivo selection of CDRs via active immunisation, and typically affinity maturation has to be done in order to improve affinity for the target antigen. Affinity maturation is a lengthy process which may add considerable time to the antibody discovery process. Also, in the process of affinity maturation certain amino acid residues may be changed which may negatively affect the binding specificity or manufacturability (e.g. expression level, solubility, physicochemical stability etc.) of the resulting antibody (Wu et al., J. Mol. Biol. 368: 652-65 (2007)).

### SUMMARY OF THE INVENTION

It remains a goal in the art to develop a technically simple platform for raising antibodies against highly conserved target antigens, which does not suffer from the problems encountered with prior art methods (e.g. low affinity, low specificity, poor diversity). In particular, it would be desirable to develop a platform for raising antibodies against highly conserved target antigens which is based on in vivo selection of CDRs via active immunisation, thereby avoiding the need for affinity maturation and the associated problems with “manufacturability” of the resulting antibody.

It has now been observed that high affinity immunologically specific antibodies against self-antigens and highly conserved target antigens can be produced by active immunisation of Camelidae species. Even using human polypeptides which display greater than 90%, and as high as 97%, amino acid sequence identity with the closest camelid homolog, it is possible to generate high specificity, high affinity antibodies by simple immunisation of the animals, without no need to generate knock-outs or otherwise manipulate either the genetic background of the animal or its natural immune function. The ability to break the barrier of self-tolerance by simple immunisation of camelids is extremely surprising and gives rise to the opportunity to raise useful antibodies, including antibodies with potential therapeutic utility, against a range of highly conserved targets, which were previously thought to be beyond the reach of the known monoclonal antibody platforms.

Accordingly, in a first aspect the invention provides an isolated antigen binding polypeptide comprising a VH domain and a VL domain, wherein at least one hypervariable loop or complementarity determining region (CDR) in the VH domain or the VL domain is obtained from a VH or VL domain of a species in the family Camelidae, characterised in that the antigen binding polypeptide specifically binds to a target antigen which is a polypeptide antigen that exhibits at least 90%, or at least 95%, 96%, 97% or 98% amino acid sequence identity with a protein from said species in the family Camelidae over a sequence comparison window that includes at least the epitope for the antigen binding polypeptide.

In one embodiment the target antigen is a polypeptide antigen that exhibits at least 90%, or at least 95%, 96%, 97% or 98% amino acid sequence identity with a protein from said species in the family Camelidae over a sequence comparison window that comprises at least one domain of the target antigen, including the epitope for the antigen binding polypeptide.

In one embodiment the target antigen is a polypeptide antigen that exhibits at least 90%, or at least 95%, 96%, 97% or 98% amino acid sequence identity with a protein from said species in the family Camelidae over the full length of the target antigen.

The target antigen may contain at least one epitope having substantially identical structure to an epitope present in the comparator protein from said species in the family Camelidae. In the case of a linear epitope, the sequence of amino acids forming the epitope may be 100% conserved between the target antigen and the comparator camelid protein.

In one embodiment the target antigen may be a highly conserved antigen, wherein said highly conserved antigen is a polypeptide from a species other than said species in the family Camelidae that exhibits at least 90%, or at least 95%, 96%, 97% or 98% amino acid sequence identity with the homologous protein from said species in the family Camelidae over a sequence comparison window that includes at least the epitope for the antigen binding polypeptide.

In one embodiment the highly conserved antigen is a polypeptide antigen that exhibits at least 90%, or at least 95%, 96%, 97% or 98% amino acid sequence identity with the homologous protein from said species in the family Camelidae over a sequence comparison window that comprises at least one domain of the target antigen, including the epitope for the antigen binding polypeptide.

In one embodiment the target antigen is a highly conserved antigen that exhibits at least 90%, or at least 95%, 96%, 97% or 98% amino acid sequence identity with the homologous protein from said species in the family Camelidae over the full length of the target antigen.

In each of the foregoing embodiments, the highly conserved antigen may contain at least one epitope having substantially identical structure to an epitope present in the comparator protein from said species in the family Camelidae.

In preferred embodiments the highly conserved antigen may be a polypeptide from a species other than said species in the family Camelidae that exhibits at least 95%, 96%, 97%, 98% or even at least 99% amino acid sequence identity with the homologous protein from said species in the family Camelidae over the sequence comparison window, as defined above, or over the full length of the target antigen.

In specific embodiments, the highly conserved antigen may be a polypeptide from a species selected from the group consisting of: human, non-human primate, mouse, rat, pig, dog, guinea pig, rabbit, sheep, cow or chicken. In particularly preferred embodiments, the highly conserved antigen is a human polypeptide.

In specific embodiments the species in the family Camelidae is selected from the group consisting of camel, llama, dromedary, vicuña, guanaco and alpaca. In a preferred embodiment the species in the family Camelidae is llama (Lama glama).

In a particularly preferred embodiment, the highly conserved antigen is a human polypeptide and the species in the family Camelidae is llama (Lama glama). In this embodiment the highly conserved antigen (i.e. the human polypeptide) may exhibit at least 90%, or at least 95%, 96%, 97%, 98%, or even at least 99% amino acid sequence identity with the homologous llama protein over a sequence comparison window as defined above.

In a further embodiment the target antigen may be a self-antigen from a species in the family Camelidae or a camelid-derived antigen which is a polypeptide antigen that exhibits at least 90%, or at least 95%, 96%, 97% or 98% amino acid sequence identity with the Camelidae self-antigen over a sequence comparison window that includes at least the epitope for the antigen binding polypeptide.

In one embodiment the target antigen is a camelid-derived antigen that exhibits at least 90%, or at least 95%, 96%, 97% or 98% amino acid sequence identity with the camelid self-antigen over a sequence comparison window that comprises at least one domain of the target antigen, including the epitope for the antigen binding polypeptide.

In one embodiment the target antigen is a camelid-derived antigen that exhibits at least 90%, or at least 95%, 96%, 97% or 98% amino acid sequence identity with the camelid self-antigen over the full length of the target antigen.

The camelid-derived antigen may retain the epitope of the camelid self-antigen from which it is derived, or it may contain at least one epitope having substantially identical structure to an epitope present in this self-antigen.

In one specific embodiment the target antigen is the variable region of an antibody obtained from said species in the family Camelidae or a sequence variant which exhibits at least 90% amino acid sequence identity therewith. In particular embodiments, the sequence variant may exhibit at least 95%, 96%, 97%, 98% or 99% amino acid sequence identity with the variable region of an antibody obtained from said species in the family Camelidae. The comparison window for sequence comparison of the variant with the variable region of an antibody obtained from said species in the family Camelidae may, in the case of camelid conventional antibodies, comprise the full length of the VH domain and the full length of the VL domain. In the case of camelid heavy-chain only antibodies containing a VHH domain, the sequence comparison window may be the full length of the VHH domain. The sequence variant may retain the epitope (or idiotype) of the camelid antibody variable domain from which it is derived.

In a second aspect the invention provides a process for preparing a recombinant antigen binding polypeptide that specifically binds to a target antigen, said antigen binding polypeptide comprising a VH domain and a VL domain, wherein at least one hypervariable loop or complementarity determining region (CDR) in the VH domain or the VL domain is obtained from a species in the family Camelidae, said process comprising the steps of:

(a) immunising a species in the family Camelidae, thereby raising a conventional antibody to said target antigen.

(b) isolating Camelidae nucleic acid encoding at least one hypervariable loop or complementarity determining region (CDR) of the VH and/or the VL domain of a Camelidae conventional antibody immunoreactive with said target antigen;

(c) preparing a polynucleotide comprising a nucleotide sequence encoding hypervariable loop(s) or complementarity determining region(s) having amino acid sequence identical to the hypervariable loop(s) or complementarity determining region(s) encoded by the nucleic acid isolated in step (a), which polynucleotide encodes an antigen binding polypeptide comprising a VH domain and a VL domain that is immunoreactive with (or specifically binds to) said target antigen; and

(d) expressing said antigen binding polypeptide from the recombinant polynucleotide of step (c), wherein said antigen binding polypeptide is not identical to the Camelidae conventional antibody of part (b), characterised in that said target antigen is a polypeptide antigen that exhibits at least 90% or at least 95%, 96%, 97% or 98% amino acid sequence identity with a protein from said species in the family Camelidae over a sequence comparison window that includes at least the epitope for the antigen binding polypeptide.

In one embodiment the target antigen is a polypeptide antigen that exhibits at least 90% or at least 95% amino acid sequence identity with a protein from said species in the family Camelidae over a sequence comparison window that comprises at least one domain of the target antigen, including the epitope for the antigen binding polypeptide.

In one embodiment the target antigen is a polypeptide antigen that exhibits at least 90% or at least 95%, 96%, 97% or 98% amino acid sequence identity with a protein from said species in the family Camelidae over the full length of the target antigen.

In a preferred embodiment of this process, the target antigen against which an antibody is raised in step (a) is a highly conserved antigen, wherein said highly conserved antigen is a polypeptide from a species other than said species in the family Camelidae that exhibits at least 90% or at least 95%, 96%, 97% or 98% amino acid sequence identity with the homologous protein from said species in the family Camelidae over a sequence comparison window as defined above.

In further preferred embodiments of the process the highly conserved antigen may be a polypeptide from a species other than said species in the family Camelidae that exhibits at least 95%, 96%, 97%, 98% or 99% amino acid sequence identity with the homologous protein from said species in the family Camelidae over a sequence comparison window as defined above.

In specific embodiments, the highly conserved antigen may be a polypeptide from a species selected from the group consisting of: human, non-human primate, mouse, rat, pig, dog, guinea pig, rabbit, sheep, cow, or chicken. In particularly preferred embodiments, the highly conserved antigen is a human polypeptide.

In specific embodiments the species in the family Camelidae which is immunised with the target antigen is selected from the group consisting of camel, llama, dromedary, vicuña, guanaco and alpaca. In a preferred embodiment the species in the family Camelidae is llama (Lama glama).

In a particularly preferred embodiment, the highly conserved antigen is a human polypeptide and the species in the family Camelidae which is immunised with said highly conserved antigen is llama (Lama glama). In this embodiment the highly conserved antigen (i.e. the human polypeptide) will exhibit at least 90%, or at least 95%, 96%, 97%, 98% or 99% amino acid sequence identity with the homologous llama protein over a sequence comparison window as defined above.

In a specific, non-limiting embodiment the target antigen may be the variable region of an antibody obtained from said species in the family Camelidae or a sequence variant which exhibits at least 90% amino acid sequence identity therewith over a sequence comparison window as defined above. In particular embodiments, the sequence variant may exhibit at least 95%, 96%, 97%, 98% or 99% amino acid sequence identity with the variable region of an antibody obtained from said species in the family Camelidae over a sequence comparison window as defined above.

### BRIEF DESCRIPTION OF THE DRAWINGS

FIG. 1 illustrates the binding properties of purified llama-derived Fabs as determined by a HMGB1 and HMGB2 ELISA.

FIG. 2 illustrates the inhibition of the HMGB1/RAGE interaction by the llama-derived Fab 2A1, as measured by ELISA.

FIG. 3 illustrates the binding profile of the llama-IgG version of anti-idiotypic antibody 11E1 (la11E1) with a panel of antibodies, assessed by ELISA.

FIG. 4 demonstrates the ability of the llama-IgG version of anti-idiotypic antibody 11E1 (la11E1) to detect target antibodies 68F2 and 129D3 in a background of cynomolgus monkey plasma.

FIG. 5 shows a Log(dose)-response curve of 7C6(Fab) binding to 61H7 (mAb) measured by ELISA in which absorption at 620 nm is plotted versus the logarithm of the concentration of 7C6 Fab, allowing calculation of EC50.

FIG. 6 illustrates binding of anti-idiotypic mAbs to 41D12 (Fab) as measured by ELISA.

### DETAILED DESCRIPTION OF THE INVENTION

The present invention has arisen from the surprising observation that it is possible to raise high affinity, high specificity antibodies against highly conserved target antigens, and even self-antigens, by active immunisation of Camelidae species. Therefore, in its broadest aspect the invention provides camelid-derived antigen binding polypeptides (i.e. antigen binding polypeptides comprising a VH domain and a VL domain, wherein at least one hypervariable loop or complementarity determining region (CDR) in the VH domain or the VL domain is obtained from a species in the family Camelidae) which are characterised by specific binding to target antigens that are highly similar in sequence and/or structure to the camelid's own antigens. The invention also provides camelid-derived antigen binding polypeptides which specifically bind to camelid self-antigens.

The general features of camelid-derived conventional antibodies are described in the applicant's own earlier publications, WO 2010/001251 and WO 2011/080350, the contents of which are incorporated herein in their entirety by reference. The heavy and light chain variable domains of camelid-derived conventional antibodies (as differentiated from the camelid heavy chain only antibodies) are characterised by their exceptionally high homology with human antibodies, both at the level of primary amino acid sequence within the framework regions of the heavy and light chain variable domains and in the three-dimensional conformation of the CDRs which contribute to the antigen binding site (discussed in extensive detail in WO 2010/001251). Moreover, the variable domains of camelid conventional antibodies can be rendered even more human-like by a simple process of “germlining” in which a small number of selected amino acids within the framework regions are substituted with homologous amino acids from the human germline (discussed extensively in WO 2011/080350).

It has now been observed that high affinity high specificity camelid conventional antibodies can be raised against a group of target antigens which were previously thought to be extremely difficult to target using known antibody development techniques, particularly using antibody platforms which rely on immunisation of a host animal. These “difficult-to-target” antigens are characterised by their very close sequence and/or structural similarity to antigens naturally expressed in the system in which the target antibody is to be raised (e.g. the host camelid) and include both self-antigens from the species of camelid in which the antibody is to be raised and also polypeptide antigens which exhibit at least 90% amino acid sequence similarity with the closest matching camelid polypeptide from the same species of camelid in which the antibody is to be raised. The antigen binding polypeptides provided herein not only exhibit high affinity specific binding to “difficult-to-target” antigens, but also retain the particular advantages of the camelid conventional antibodies, namely the extremely high sequence homology and structural homology with the variable domains of human antibodies.

Definitions

The term “antigen binding polypeptide” refers to any polypeptide comprising a VH domain and a VL domain which is immunoreactive with, exhibits specific binding to, a target antigen. Exemplary antigen binding polypeptides include antibodies and immunoglobulins, and also antibody fragments, as discussed elsewhere herein.

The term “target antigen” refers to the antigen against which the antigen binding polypeptide is immunoreactive.

The terms “target antigen” or “antigenic material” can also be used to describe the material employed in the immunisation of animals (e.g. camelids) during the manufacture of antigen binding polypeptides reactive with the target antigen of interest. In this context the term “target antigen” could encompass purified forms of the antigen, and also crude or semi-purified preparations of the antigen, such as for example cells, cell lysates or supernatants, cell fractions, e.g. cell membranes, etc., plus haptens conjugated with an appropriate carrier protein, or a truncated form of the target antigen, e.g. a fragment containing an immunogenic epitope, or even a polynucleotide capable of encoding the target antigen, as would be used for “DNA immunisation”. Further characteristics of “target antigens” or “antigenic materials” used for active immunisation of camelids are described elsewhere herein, and would be generally known to a person skilled in the art.

“Specific binding” between and antigen binding polypeptide and a target antigen refers to immunological specificity. An antigen binding polypeptide binds “specifically” to its target antigen if it binds an epitope on the target antigen in preference to other epitopes. “Specific binding” does not exclude cross-reactivity with other antigens bearing similar antigenic epitopes.

“Antibodies” (Abs) and “immunoglobulins” (Igs) are glycoproteins which exhibit binding specificity to a (target) antigen.

The camelid species are known to possess two different types of antibodies; the classical or “conventional” antibodies and also the heavy-chain antibodies.

As used herein, the term “conventional antibody” refers to antibodies of any isotype, including IgA, IgG, IgD, IgE or IgM. Native or naturally occurring “conventional” camelid antibodies are usually heterotetrameric glycoproteins, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end (N-terminal) a variable domain (VH) followed by a number of constant domains. Each light chain has a variable domain (VL) at one end (N-terminal) and a constant domain (CL) at its other end; the constant domain of the light chain is aligned with the first constant domain of the heavy chain, and the light-chain variable domain is aligned with the variable domain of the heavy chain. Particular amino acid residues are believed to form an interface between the light- and heavy-chain variable domains.

The term “heavy-chain antibody” refers to the second type of antibodies known to occur naturally in camelid species, such antibodies being naturally devoid of light chains (Hamers-Casterman, et al. Nature. 1993; 363; 446-8). The heavy-chain antibodies (abbreviated to HCAb) are composed of two heavy chains linked by a covalent disulphide bond. Each heavy chain in the HCAb has a variable domain at one end. The variable domains of HCAbs are referred to as “VHH” in order to distinguish them from the variable domains of the heavy chains of “conventional” camelid antibodies (VH). The VHH domains and VH domains are entirely distinct and are encoded by different gene segments in the camelid genome.

The VL domains in the polypeptide of the invention may be of the VLambda type or the Vkappa type. The term “VL domain” therefore refers to both VKappa and VLambda isotypes from Camelidae, and engineered variants thereof which contain one or more amino acid substitutions, insertions or deletions relative to a Camelidae VL domain.

The term “VH domain” refers to a VH domain of any known heavy chain isotype of Camelidae, including γ, ε, δ, α or μ isotypes, as well as engineered variants thereof which contain one or more amino acid substitutions, insertions or deletions relative to a Camelidae VH domain. The term “VH domain” refers only to VH domains of camelid conventional antibodies and does not encompass camelid VHH domains.

The term “variable” refers to the fact that certain portions of the variable domains VH and VL differ extensively in sequence among antibodies and are used in the binding and specificity of each particular antibody for its target antigen. However, the variability is not evenly distributed throughout the variable domains of antibodies. It is concentrated in three segments called “hypervariable loops” in each of the VL domain and the VH domain which form part of the antigen binding site. The first, second and third hypervariable loops of the VLambda light chain domain are referred to herein as L1(λ), L2(λ) and L3(λ) and may be defined as comprising residues 24-33 (L1(λ), consisting of 9, 10 or 11 amino acid residues), 49-53 (L2(λ), consisting of 3 residues) and 90-96 (L3(λ), consisting of 5 residues) in the VL domain (Morea et al., Methods 20:267-279 (2000)). The first, second and third hypervariable loops of the VKappa light chain domain are referred to herein as L1(κ), L2(κ) and L3(κ) and may be defined as comprising residues 25-33 (L1(κ), consisting of 6, 7, 8, 11, 12 or 13 residues), 49-53 (L2(κ), consisting of 3 residues) and 90-97 (L3(κ), consisting of 6 residues) in the VL domain (Morea et al., Methods 20:267-279 (2000)). The first, second and third hypervariable loops of the VH domain are referred to herein as H1, H2 and H3 and may be defined as comprising residues 25-33 (H1, consisting of 7, 8 or 9 residues), 52-56 (H2, consisting of 3 or 4 residues) and 91-105 (H3, highly variable in length) in the VH domain (Morea et al., Methods 20:267-279 (2000)).

Unless otherwise indicated, the terms L1, L2 and L3 respectively refer to the first, second and third hypervariable loops of a VL domain, and encompass hypervariable loops obtained from both Vkappa and Vlambda isotypes from Camelidae. The terms H1, H2 and H3 respectively refer to the first, second and third hypervariable loops of the VH domain, and encompass hypervariable loops obtained from any of the known heavy chain isotypes from Camelidae, including γ, ε, δ, α or μ.

The hypervariable loops L1, L2, L3, H1, H2 and H3 may each comprise part of a “complementarity determining region” or “CDR”. The terms “hypervariable loop” and “complementarity determining region” are not strictly synonymous, since the hypervariable loops (HVs) are defined on the basis of structure, whereas complementarity determining regions (CDRs) are defined based on sequence variability (Kabat et al., Sequences of Proteins of Immunological Interest, 5th Ed. Public Health Service, National Institutes of Health, Bethesda, Md., 1983) and the limits of the HVs and the CDRs may be different in some VH and VL domains.

The CDRs of the VL and VH domains can typically be defined as comprising the following amino acids: residues 24-34 (CDRL1), 50-56 (CDRL2) and 89-97 (CDRL3) in the light chain variable domain, and residues 31-35 or 31-35b (CDRH1), 50-65 (CDRH2) and 95-102 (CDRH3) in the heavy chain variable domain; Kabat et al., Sequences of Proteins of Immunological Interest, 5th Ed. Public Health Service, National Institutes of Health, Bethesda, Md. (1991)). Thus, the HVs may be comprised within the corresponding CDRs and references herein to the “hypervariable loops” of VH and VL domains should be interpreted as also encompassing the corresponding CDRs, and vice versa, unless otherwise indicated.

The more highly conserved portions of variable domains are called the framework region (FR). The variable domains of native heavy and light chains each comprise four FRs (FR1, FR2, FR3 and FR4, respectively), largely adopting a β-sheet configuration, connected by the three hypervariable loops. The hypervariable loops in each chain are held together in close proximity by the FRs and, with the hypervariable loops from the other chain, contribute to the formation of the antigen-binding site of antibodies. Structural analysis of antibodies revealed the relationship between the sequence and the shape of the binding site formed by the complementarity determining regions (Chothia et al., J. Mol. Biol. 227: 799-817 (1992)); Tramontano et al., J. Mol. Biol, 215:175-182 (1990)). Despite their high sequence variability, five of the six loops adopt just a small repertoire of main-chain conformations, called “canonical structures”. These conformations are first of all determined by the length of the loops and secondly by the presence of key residues at certain positions in the loops and in the framework regions that determine the conformation through their packing, hydrogen bonding or the ability to assume unusual main-chain conformations.

The constant domains are not involved directly in binding of an antibody to an antigen, but exhibit various effector functions, such as participation of the antibody in antibody-dependent cellular toxicity (ADCC) or complement-dependent cytotoxicity (CDC).

In all aspects and embodiments of the invention, the Camelidae (or camelid) species (from which the hypervariable loops or CDRs of the antigen binding polypeptide of the invention are obtained) can be camel, llama, dromedary, vicuña, guanaco or alpaca and any crossings thereof. Llama (Lama glama) and alpaca (Lama pacos) are the preferred Camelidae species for all aspects of the invention.

Antigen binding polypeptides are considered to be “camelid-derived” if they contain at least one hypervariable loop or complementarity determining region which is obtained from a VH domain or a VL domain of a species in the family Camelidae. For the avoidance of doubt, the terms “VH domain” “VL domain” refer to domains derived from camelid conventional antibodies. This definition excludes the camelid heavy chain only VHH antibodies, and recombinant constructs containing solely HVs or CDRs of camelid VHH domains, which are not encompassed within the scope of the present invention.

By “hypervariable loop or complementarity determining region obtained from a VH domain or a VL domain of a species in the family Camelidae” is meant that that hypervariable loop (HV) or CDR has an amino acid sequence which is identical, or substantially identical, to the amino acid sequence of a hypervariable loop or CDR which is encoded by a Camelidae immunoglobulin gene. In this context “immunoglobulin gene” includes germline genes, immunoglobulin genes which have undergone rearrangement, and also somatically mutated genes. Thus, the amino acid sequence of the HV or CDR obtained from a VH or VL domain of a Camelidae species may be identical to the amino acid sequence of a HV or CDR present in a mature Camelidae conventional antibody. The term “obtained from” in this context implies a structural relationship, in the sense that the HVs or CDRs of the antigen binding polypeptide of the invention embody an amino acid sequence (or minor variants thereof) which was originally encoded by a Camelidae immunoglobulin gene. However, this does not necessarily imply a particular relationship in terms of the production process used to prepare the antigen binding polypeptide of the invention. As will be discussed below, there are several processes which may be used to prepare antigen binding polypeptides comprising HVs or CDRs with amino acid sequences identical to (or substantially identical to) sequences originally encoded by a Camelidae immunoglobulin gene.

The terms “VH domain of a conventional antibody of a camelid” and “VH domain obtained from a species of Camelidae” are used synonymously and encompass VH domains which are the products of synthetic or engineered recombinant genes (including codon-optimised synthetic genes), which VH domains have an amino acid sequence identical to (or substantially identical to) the amino acid sequence of a VH domain encoded by a Camelidae immunoglobulin gene (germline, rearranged or somatically mutated). Similarly, the terms “VL domain of a conventional antibody of a camelid” and “VL domain obtained from a species of Camelidae ” are used synonymously and encompass VL domains which are the products of synthetic or engineered recombinant genes (including codon-optimised synthetic genes), which VL domains have an amino acid sequence identical to (or substantially identical to) the amino acid sequence of a VL domain encoded by a Camelidae immunoglobulin gene (germline, rearranged or somatically mutated).

The antigen binding polypeptides provided herein are typically recombinantly expressed polypeptides, and may be chimeric polypeptides. The term “chimeric polypeptide” refers to an artificial (non-naturally occurring) polypeptide which is created by juxtaposition of two or more peptide fragments which do not otherwise occur contiguously. Included within this definition are “species” chimeric polypeptides created by juxtaposition of peptide fragments encoded by two or more species, e.g. camelid and human.

The antigen binding provided herein are not naturally occurring human antibodies, specifically human autoantibodies, due to the requirement for at least one hypervariable loop (or CDR) from camelid. By “naturally occurring” human antibody is meant an antibody which is naturally expressed within a human subject. Antigen binding polypeptides having an amino acid sequence which is 100% identical to the amino acid sequence of a naturally occurring human antibody, or a fragment thereof, which natural antibody or fragment is not chimeric and has not been subject to any engineered changes in amino acid sequence (excluding somatic mutations) are excluded from the scope of the invention.

The antigen binding polypeptides provided herein comprise both a heavy chain variable (VH) domain and a light chain variable (VL) domain, and are characterised in that at least one hypervariable loop or complementarity determining region in either the VH domain or the VL domain is obtained from a species in the family Camelidae.

In alternative embodiments, either H1 or H2, or both H1 and H2 in the VH domain may be obtained from a species in the family Camelidae, and independently either L1 or L2 or both L1 and L2 in the VL domain may be obtained from a species in the family Camelidae. In further embodiments H3 in the VH domain or L3 in the VL domain may also be obtained from a species in the family Camelidae. All possible permutations of the foregoing are permitted. In one specific embodiment each of the hypervariable loops H1, H2, H3, L1, L2 and L3 in both the VH domain and the VL domain may be obtained from a species in the family Camelidae.

In one embodiment the entire VH domain and/or the entire VL domain may be obtained from a species in the family Camelidae. The Camelidae VH domain and/or the Camelidae VL domain may then be subject to protein engineering, in which one or more amino acid substitutions, insertions or deletions are introduced into the Camelidae sequence. These engineered changes preferably include amino acid substitutions relative to the Camelidae sequence. Such changes include “humanisation” or “germlining” wherein one or more amino acid residues in a camelid-encoded VH or VL domain are replaced with equivalent residues from a homologous human-encoded VH or VL domain.

In certain embodiments, Camelidae hypervariable loops (or CDRs) may be obtained by active immunisation of a species in the family Camelidae with an antigenic material which is capable of eliciting antibodies reactive with the desired “target antigen” of interest. As discussed and exemplified in detail herein, following immunisation of Camelidae (either the native animal or a transgenic animal engineered to express the immunoglobulin repertoire of a camelid species) with antigenic material capable of eliciting antibodies immunoreactive with the target antigen, B cells producing (conventional Camelidae) antibodies having specificity for the desired antigen can be identified and polynucleotide encoding the VH and VL domains of such antibodies can be isolated using known techniques.

Thus, in a specific embodiment, the invention provides a recombinant antigen binding polypeptide immunoreactive with a target antigen, the polypeptide comprising a VH domain and a VL domain, wherein at least one hypervariable loop or complementarity determining region in the VH domain or the VL domain is obtained from a VH or VL domain of a species in the family Camelidae, and characterised in that the antigen binding polypeptide specifically binds to a target antigen which is a polypeptide antigen that exhibits at least 90% amino acid sequence identity with a protein from said species in the family Camelidae over a sequence comparison window that includes at least the epitope for the antigen binding polypeptide, which antigen binding polypeptide is obtainable by a process comprising the steps of:

(a) immunising a species in the family Camelidae and thereby raising an antibody to said target antigen;

(b) determining the nucleotide sequence encoding at least one hypervariable loop or complementarity determining region (CDR) of the VH and/or the VL domain of a Camelidae conventional antibody immunoreactive with said target antigen; and

(c) expressing an antigen binding polypeptide immunoreactive with said target antigen, said antigen binding polypeptide comprising a VH and a VL domain, wherein at least one hypervariable loop or complementarity determining region (CDR) of the VH domain or the VL domain has an amino acid sequence encoded by the nucleotide sequence determined in part (a).

Isolated Camelidae VH and VL domains obtained by active immunisation can be used as a basis for engineering antigen binding polypeptides according to the invention. Starting from intact Camelidae VH and VL domains, it is possible to engineer one or more amino acid substitutions, insertions or deletions which depart from the starting Camelidae sequence. In certain embodiments, such substitutions, insertions or deletions may be present in the framework regions of the VH domain and/or the VL domain. The purpose of such changes in primary amino acid sequence may be to reduce presumably unfavourable properties (e.g. immunogenicity in a human host (so-called humanization), sites of potential product heterogeneity and or instability (glycosylation, deamidation, isomerisation, etc.) or to enhance some other favourable property of the molecule (e.g. solubility, stability, bioavailability, etc.). In other embodiments, changes in primary amino acid sequence can be engineered in one or more of the hypervariable loops (or CDRs) of a Camelidae VH and/or VL domain obtained by active immunisation. Such changes may be introduced in order to enhance antigen binding affinity and/or specificity, or to reduce presumably unfavourable properties, e.g. immunogenicity in a human host (so-called humanization or germlining), sites of potential product heterogeneity and or instability, glycosylation, deamidation, isomerisation, removal of sulphur-containing amino acids such as methionine etc., or to enhance some other favourable property of the molecule, e.g. solubility, stability, bioavailability, manufacturability etc.

Thus, in one embodiment, the invention provides a recombinant antigen binding polypeptide which contains at least one amino acid substitution in at least one framework or CDR region of either the VH domain or the VL domain in comparison to a Camelidae VH or VL domain obtained by active immunisation of a species in the family Camelidae with a target antigen, characterised in that the antigen binding polypeptide specifically binds to a target antigen which is a polypeptide antigen that exhibits at least 90% amino acid sequence identity with a protein from said species in the family Camelidae. This particular embodiment excludes antigen binding polypeptides containing native Camelidae VH and VL domains produced by active immunisation

In other embodiments, the invention encompasses “chimeric” antibody molecules comprising VH and VL domains from Camelidae (or engineered variants thereof) and one or more constant domains from a non-camelid antibody, for example human-encoded constant domains (or engineered variants thereof). In such embodiments it is preferred that both the VH domain and the VL domain are obtained from the same species of camelid, for example both VH and VL may be from Lama glama or both VH and VL may be from Lama pacos (prior to introduction of engineered amino acid sequence variation). In such embodiments both the VH and the VL domain may be derived from a single animal, particularly a single animal which has been actively immunised. The invention also extends to chimeric antigen binding polypeptides (e.g. antibody molecules) wherein one of the VH or the VL domain is camelid-encoded, and the other variable domain is non-camelid (e.g. human).

As an alternative to engineering changes in the primary amino acid sequence of Camelidae VH and/or VL domains, individual Camelidae hypervariable loops or CDRs, or combinations thereof, can be isolated from Camelidae VH/VL domains and transferred to an alternative (i.e. non-Camelidae) framework, e.g. a human VH/VL framework, by CDR grafting.

### Antigen Binding Polypeptides Binding Highly Conserved Antigens

In one preferred embodiment of the invention the target antigen that exhibits at least 90% amino acid sequence identity with a protein from said species in the family Camelidae may be a “highly conserved” antigen. In the case of antigen binding polypeptides raised by active immunisation of a camelid species, a target antigen is considered “highly conserved” if it is extremely similar in sequence and/or structure to a native antigen of the same camelid species as that in which the antigen binding polypeptide is/was raised. For example, in the case of antigen binding polypeptides which are llama-derived, the “highly conserved” target antigen is a polypeptide from a species other than llama which is highly similar to a native llama antigen.

In the case of polypeptide antigens, the degree of similarity between the highly conserved antigen and the native camelid polypeptide to which it is most closely related in structure may be expressed in terms of % similarity of amino acid sequences. Therefore, in preferred embodiments the highly conserved antigen is a polypeptide from a species other than said species in the family Camelidae that exhibits at least 90% amino acid sequence identity with the homologous protein from said species in the family Camelidae. In further preferred embodiments the highly conserved antigen may be a polypeptide from a species other than said species in the family Camelidae that exhibits at least 95%, 96%, 97%, 98% or 99% amino acid sequence identity with the homologous protein from said species in the family Camelidae.

Unless otherwise stated in the present application, % sequence identity between two amino acid sequences may be determined by comparing these two sequences aligned in an optimum manner and in which the amino acid sequence to be compared can comprise additions or deletions with respect to the reference sequence for an optimum alignment between these two sequences. The percentage of identity is calculated by determining the number of positions for which the amino acid residue is identical between the two sequences, by dividing this number of identical positions by the total number of positions in the chosen sequence comparison window and by multiplying the result obtained by 100 in order to obtain the percentage of identity between these two sequences. For example, it is possible to use the BLAST program, “BLAST 2 sequences” (Tatusova et al, “Blast 2 sequences—a new tool for comparing protein and nucleotide sequences”, FEMS Microbiol Lett. 174:247-250) available on the site http://www.ncbi.nlm.nih.gov/ gorf/bl2.html, the parameters used being those given by default (in particular for the parameters “open gap penalty”: 5, and “extension gap penalty”: 2; the matrix chosen being, for example, the matrix “BLOSUM 62” proposed by the program), the percentage of identity between the two sequences to be compared being calculated directly by the program.

For any given target antigen, it would also be possible for the skilled person to use publicly available search and alignment tools, such as the BLAST program, with the target antigen sequence as a single query sequence. From the results returned, it would be possible to determine whether or not the polypeptide antigen exhibits at least 90%, 95%, 96%, 97%, 98% or 99% amino acid sequence identity with a protein from the relevant Camelidae species i.e. the Camelidae species from which the hypervariable loop or complementarity determining region of the antigen binding polypeptide derives.

In all aspects and embodiments of the invention, the “sequence comparison window” over which the target antigen is compared to the comparator camelid protein must be a region of the target antigen that includes at least the epitope for the antigen binding polypeptide. The sequence comparison may be, for example, a contiguous stretch of at least 20 amino acids, or at least 30 amino acids, or at least 50 amino acids, or at least 100 amino acids of the target protein, which includes the epitope for the antigen binding polypeptides.

In certain embodiments the sequence comparison window may comprise at least one domain of the target antigen, including the epitope for the antigen binding polypeptide.

In further embodiments the sequence comparison window may be the full length of the target antigen which, by definition, will include the epitope for the antigen binding polypeptide, even if the precise identity/location of the epitope is not known.

In each of the foregoing embodiments the target antigen may contain an epitope (for the antigen binding polypeptide) having substantially identical structure to an epitope present in the comparator protein from said species in the family Camelidae. In the case of a linear epitope, the sequence of amino acids forming the epitope may be 100% conserved between the target antigen and the comparator camelid protein.

The highly conserved antigen may be a polypeptide from essentially any species, including but not limited to human, non-human primate, mouse, rat, pig, dog, guinea pig, rabbit, cow or chicken. However, in particularly preferred embodiments the highly conserved antigen may be a human polypeptide, e.g. a therapeutic target or biomarker. In particularly preferred embodiments the highly conserved antigen may be a target antigen of therapeutic or diagnostic relevance. The precise nature of the human polypeptide is not material; the present invention provides a platform for raising antibodies against human polypeptides that are “highly conserved” irrespective of the precise identity of the human polypeptide. The human polypeptides (against which antigen binding polypeptides of the invention can be raised) are characterised by their degree of similarity (in amino acid sequence) to native camelid antigens. By way of non-limiting example, one “highly conserved” human polypeptide against which antibodies can be raised is the high mobility group box 1 (HMGB1) protein, which is 99% conserved between human and mouse and 97% conserved between human and llama.

In other embodiments the highly conserved antigen may be a non-polypeptide antigen, for example a carbohydrate antigen (e.g. an oligosaccharide or polysaccharide), or an antigen comprising both polypeptide and carbohydrate components, e.g. a glycoprotein. In the case of a target antigen which is a glycoprotein, the glycoprotein target antigen may exhibit a glycosylation pattern which is substantially identical to the glycosylation pattern of the homologous camelid protein. The antigenic determinant to which the antigen-binding molecule binds may be a carbohydrate epitope or it may be an epitope formed by a combination of polypeptide determinants (e.g. one or more amino acid residues) plus carbohydrate determinants (e.g. one or more sugar moieties of an oligosaccharide/glycan side chain of the glycoprotein).

### Antigen Binding Polypeptides Binding Camelid Self-Antigens and Camelid-Derived Antigens

In a second preferred embodiment of the invention the target antigen that exhibits at least 90% amino acid sequence identity with a protein from said species in the family Camelidae may be a camelid self-antigen or a camelid-derived antigen. In the case of antigen binding polypeptides raised by active immunisation of a camelid species, the term “self-antigen” refers to a native camelid antigen, i.e. an antigen which occurs naturally in the same camelid species. For example, in the case of antigen binding polypeptides raised by active immunisation of llama, the term “self-antigen” refers to a native llama antigen, i.e. an antigen which occurs naturally in the llama.

Camelid (e.g. llama) self-antigens may include, inter alia, polypeptide, peptide, polysaccharide, glycoprotein, polynucleotide (e.g. DNA), antigens, with polypeptide self-antigens being particularly preferred. “Polypeptide self-antigens” may include natural camelid polypeptides and also synthetic versions of camelid polypeptides, for example recombinantly expressed polypeptides.

The term “camelid-derived antigen” is used herein to refer to a target antigen which is a variant derived from a native camelid antigen, said variant being highly similar in structure/sequence to the camelid antigen, for example engineered sequence variants of a camelid polypeptide. A camelid-derived polypeptide antigen may have an amino acid sequence which is highly similar to the amino acid sequence of the native camelid protein from which it is/was derived, for example an amino acid sequence at least 90%, or at least 95%, 96%, 97% or 98%, or even at least 99% identical to a native camelid polypeptide.

### Anti-Idiotypes

In one particularly important embodiment, the antigen binding polypeptide may be an anti-idiotype antigen binding polypeptide (e.g. an anti-idiotype antibody or an antigen binding fragment thereof) which binds to a target antigen comprising the variable region of an antibody obtained from a species in the family Camelidae. The camelid-derived antibody (to which the anti-idiotype binds) may be a convention camelid antibody (i.e. a camelid antibody comprising paired VH and VL domains) or a heavy-chain only antibody (i.e. a camelid antibody comprising a VHH domain). The camelid species in which the anti-idiotype antigen binding polypeptide is raised may be the same as the camelid species from which the antibody variable region is/was derived. Accordingly, by way of non-limiting example, one preferred embodiment is a llama-derived anti-idiotype antigen binding polypeptide (e.g. a llama anti-idiotype antibody) which binds to an epitope within the variable region of a llama-derived antibody (the target antigen). The llama-derived antibody which forms the target antigen for the anti-idiotype antibody may be a convention llama antibody or a heavy-chain only llama antibody.

The epitope in the variable region of the camelid-derived (e.g. llama-derived) antibody to which the anti-idiotype antigen binding polypeptide binds may be located in the VH domain of the camelid-derived (e.g. llama-derived) conventional antibody, or within the VL of the camelid-derived (e.g. llama-derived) conventional antibody, or the epitope may be formed from amino acids within both the VH domain and the VL domain of the camelid-derived (e.g. llama-derived) conventional antibody. The “epitope” for an anti-idiotype antigen binding polypeptide is most typically formed from the CDRs of the antibody variable region to which it binds.

The variable region of the camelid-derived (e.g. llama-derived) conventional antibody which forms the target antigen for the anti-idiotype antigen binding polypeptide may be derived from a native camelid (e.g. llama) conventional antibody, for example an antibody raised by active immunisation of the camelid (e.g. llama) with an antigen of interest, or it may in fact be a synthetic or engineered sequence variant of a native camelid (e.g. llama) conventional antibody. Accordingly, in this context, the “variable regions of conventional camelid-derived antibodies” include not only the variable regions of native camelid conventional antibodies, i.e. having identical sequence to antibodies raised in the camelid, but also encompasses engineered variants, for example variants with amino acid substitutions in the framework regions and/or the CDRs relative to the native camelid sequence, provided that the engineered variant should still retain a minimum of at least 90% amino acid sequence identity with the variable domains (VH and/or VL) of the native camelid-derived conventional antibody. In such embodiments the sequence comparison window for assessment of % amino acid sequence identity may include the entire VH domain and the entire VL domain.

WO 2010/001251 describes the use of camelids (and in particular llamas) as a platform for raising conventional, four-chain, antibodies against a range of target antigens, including human polypeptide targets of therapeutic interest. Described therein are a number of techniques for raising conventional camelid antibodies against target antigens of interest. Once a native camelid (e.g. llama) conventional antibody with appropriate binding specificity for the target antigen has been isolated, it is typical to engineer one or more changes in primary amino acid sequence within the variable domains of the native camelid antibody in order to improve it's properties, for example to render it more suitable for human therapeutic use. Such changes can include amino acid substitutions within the framework regions of the VH domain and/or the VL domain and also amino acid substitutions within one or more of the CDRs within the VH and/or the VL domains that contribute to the antigen binding site. As noted above, the “epitope” of an anti-idiotype antigen binding polypeptide is most typically formed from the CDRs of the antibody variable region to which it binds. Accordingly, an anti-idiotype antigen binding polypeptide raised by active immunisation of a camelid species (e.g. llama) with the variable regions of a target antibody from the same species (e.g. immunisation with a llama Fab) is also expected to bind a germlined variant of those variable regions (e.g. germlined version of the llama Fab), particularly if the amino acid substitutions introduced for the purposes of “germlining” are confined to the framework regions, leaving the CDRs essentially unchanged in terms of sequence and structural conformation.

The present invention now provides a means by which to raise high specificity, high affinity anti-idiotype antigen binding polypeptides with binding specificity for the variable regions of conventional camelid-derived antibodies, even though the latter could be considered camelid self-antigens which are difficult to target due to immune tolerance.

An important practical application of anti-idiotype antigen binding polypeptides as described herein is as a tool for pharmacokinetic studies on camelid-derived antibodies intended for use as human therapeutic agents.

### Structure of the Antigen Binding Polypeptide

The antigen binding polypeptide of the invention can take various different embodiments, provided that both a VH domain and a VL domain are present. Thus, in non-limiting embodiments the antigen binding polypeptide may be an immunoglobulin, an antibody or antibody fragment. The term “antibody” herein is used in the broadest sense and encompasses, but is not limited to, monoclonal antibodies (including full length monoclonal antibodies), polyclonal antibodies, multispecific antibodies (e.g., bispecific antibodies), so long as they exhibit the appropriate specificity for a target antigen. The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes) on the antigen, each monoclonal antibody is directed against a single determinant or epitope on the antigen.

“Antibody fragments” comprise a portion of a full length antibody, generally the antigen binding or variable domain thereof. Examples of antibody fragments include Fab, Fab′, F(ab′)2, bi-specific Fab′s, and Fv fragments, diabodies, linear antibodies, single-chain antibody molecules, a single chain variable fragment (scFv) and multispecific antibodies formed from antibody fragments (see Holliger and Hudson, Nature Biotechnol. 23:1126-36 (2005), the contents of which are incorporated herein by reference).

In non-limiting embodiments, antibodies and antibody fragments according to the invention may comprise CH1 domains and/or CL domains, the amino acid sequence of which is fully or substantially human. Where the antigen binding polypeptide of the invention is an antibody intended for human therapeutic use, it is typical for the entire constant region of the antibody, or at least a part thereof, to have fully or substantially human amino acid sequence. Therefore, an antibody of the invention must comprise VH and VL domains, at least one of which includes at least one hypervariable loop derived from Camelidae, but one or more or any combination of the CH1 domain, hinge region, CH2 domain, CH3 domain and CL domain (and CH4 domain if present) may be fully or substantially human with respect to it's amino acid sequence.

Advantageously, the CH1 domain, hinge region, CH2 domain, CH3 domain and CL domain (and CH4 domain if present) may all have fully or substantially human amino acid sequence. In the context of the constant region of a humanised or chimeric antibody, or an antibody fragment, the term “substantially human” refers to an amino acid sequence identity of at least 90%, or at least 95%, or at least 97%, or at least 99% with a human constant region. The term “human amino acid sequence” in this context refers to an amino acid sequence which is encoded by a human immunoglobulin gene, which includes germline, rearranged and somatically mutated genes. The invention also contemplates polypeptides comprising constant domains of “human” sequence which have been altered, by one or more amino acid additions, deletions or substitutions with respect to the human sequence.

As discussed elsewhere herein, it is contemplated that one or more amino acid substitutions, insertions or deletions may be made within the constant region of the heavy and/or the light chain, particularly within the Fc region. Amino acid substitutions may result in replacement of the substituted amino acid with a different naturally occurring amino acid, or with a non-natural or modified amino acid. Other structural modifications are also permitted, such as for example changes in glycosylation pattern (e.g. by addition or deletion of N- or O-linked glycosylation sites). Depending on the intended use of the antibody, it may be desirable to modify the antibody of the invention with respect to its binding properties to Fc receptors, for example to modulate effector function. For example cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp. Med. 176:1191-1195 (1992) and Shopes, B. J. Immunol. 148:2918-2922 (1992). Alternatively, an antibody can be engineered which has dual Fc regions and may thereby have enhanced complement lysis and ADCC capabilities. See Stevenson et al., Anti-Cancer Drug Design 3:219-230 (1989). The invention also contemplates immunoconjugates comprising an antibody as described herein conjugated to a cytotoxic agent such as a chemotherapeutic agent, toxin (e.g., an enzymatically active toxin of bacterial, fungal, plant or animal origin, or fragments thereof), or a radioactive isotope (i.e., a radioconjugate). Fc regions may also be engineered for half-life extension.

The invention can, in certain embodiments, encompass chimeric Camelidae/human antibodies that specifically bind to a target antigen which is a self-antigen from said species in the family Camelidae or a polypeptide antigen that exhibits at least 90% amino acid sequence identity with a protein from said species in the family Camelidae. Preferred embodiments include chimeric antibodies in which the VH and VL domains are of fully camelid sequence (e.g. Llama or alpaca) and the remainder of the antibody is of fully human sequence. In preferred embodiments the invention also encompasses “germlined” Camelidae antibodies, and Camelidae/human chimeric antibodies, in which the VH and VL domains contain one or more amino acid substitutions in the framework regions in comparison to Camelidae VH and VL domains obtained by active immunisation. The process of “germlining” (as described in WO 2010/001251 and WO 2011/080350, incorporated herein by reference) increases the % sequence identity with human germline VH or VL domains by replacing mis-matched amino acid residues in a starting Camelidae VH or VL domain with the equivalent residue found in a human germline-encoded VH or VL domain.

The invention still further encompasses CDR-grafted antibodies in which CDRs (or hypervariable loops) derived from a Camelidae antibody that specifically binds to a target antigen which is a self-antigen from said species in the family Camelidae or a polypeptide antigen that exhibits at least 90% amino acid sequence identity with a protein from said species in the family Camelidae, for example an Camelidae antibody raised by active immunisation with a target antigen, or otherwise encoded by a camelid gene, are grafted onto a human VH and VL framework, with the remainder of the antibody also being of fully human origin

Germlined, chimeric and CDR-grafted antibodies according to the invention, particularly antibodies comprising hypervariable loops derived from active immunisation of Camelidae with a target antigen, can be readily produced using conventional recombinant DNA manipulation and expression techniques, making use of prokaryotic and eukaryotic host cells engineered to produce the polypeptide of interest and including but not limited to bacterial cells, yeast cells, mammalian cells, insect cells, plant cells, some of them as described herein and illustrated in the accompanying examples.

The invention still further extends to antigen binding polypeptides wherein the hypervariable loop(s) or CDR(s) of the VH domain and/or the VL domain are obtained from Camelidae, but wherein at least one of said (camelid-derived) hypervariable loops or CDRS has been engineered to include one or more amino acid substitutions, additions or deletions relative to the camelid-encoded sequence. Such changes include “humanisation” of the hypervariable loops/CDRs. Camelid-derived HVs/CDRs which have been engineered in this manner may still exhibit an amino acid sequence which is “substantially identical” to the amino acid sequence of a camelid-encoded HV/CDR. In this context, “substantial identity” may permit no more than one, or no more than two amino acid sequence mis-matches with the camelid-encoded HV/CDR.

Antibodies according to the invention, that specifically bind to a target antigen which is a polypeptide antigen that exhibits at least 90% amino acid sequence identity with a protein from said species in the family Camelidae, may be of any isotype. Antibodies intended for human therapeutic use will typically be of the IgA, IgD, IgE IgG, IgM type, often of the IgG type, in which case they can belong to any of the four sub-classes IgG1, IgG2a and b, IgG3 or IgG4. Within each of these sub-classes it is permitted to make one or more amino acid substitutions, insertions or deletions within the Fc portion, or to make other structural modifications, for example to enhance or reduce Fc-dependent functionalities.

Antigen binding polypeptides that specifically bind to a target antigen which is a polypeptide antigen that exhibits at least 90% amino acid sequence identity with a protein from said species in the family Camelidae may be useful in a wide range of applications, both in research and in the diagnosis and/or treatment of diseases. Because of the high degree of amino acid sequence identity with the VH and VL domains of natural human antibodies, and the high degree of structural homology (specifically the correct combinations of canonical folds as are found in human antibodies) the antigen binding polypeptides of the invention, particularly in the form of monoclonal antibodies, will find particular utility as human therapeutic agents.

### Polynucleotides, Vectors and Recombinant Expression

The invention also provides a polynucleotide molecule encoding the antigen binding polypeptide of the invention, an expression vector containing a nucleotide sequence encoding the antigen binding polypeptide of the invention operably linked to regulatory sequences which permit expression of the antigen binding polypeptide in a host cell or cell-free expression system, and a host cell or cell-free expression system containing this expression vector.

Polynucleotide molecules encoding the antigen binding polypeptide of the invention include, for example, recombinant DNA molecules.

The terms “nucleic acid”, “polynucleotide” or a “polynucleotide molecule” as used herein interchangeably and refer to any DNA or RNA molecule, either single- or double-stranded and, if single-stranded, the molecule of its complementary sequence. In discussing nucleic acid molecules, a sequence or structure of a particular nucleic acid molecule may be described herein according to the normal convention of providing the sequence in the 5′ to 3′ direction. In some embodiments of the invention, nucleic acids or polynucleotides are “isolated.” This term, when applied to a nucleic acid molecule, refers to a nucleic acid molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or non-human host organism. When applied to RNA, the term “isolated polynucleotide” refers primarily to an RNA molecule encoded by an isolated DNA molecule as defined above. Alternatively, the term may refer to an RNA molecule that has been purified/separated from other nucleic acids with which it would be associated in its natural state (i.e., in cells or tissues). An isolated polynucleotide (either DNA or RNA) may further represent a molecule produced directly by biological or synthetic means and separated from other components present during its production.

Standard techniques for recombinant production of an antigen binding polypeptide according to the invention are described in WO 2010/001251 and WO 2011/080350, the contents of which are incorporated herein entirely by reference.

It should be noted that the term “host cell” generally refers to a cultured cell line. Whole human beings into which an expression vector encoding an antigen binding polypeptide according to the invention has been introduced are explicitly excluded from the scope of the invention.

In an important aspect, the invention also provides a method of producing a recombinant antigen binding polypeptide which comprises culturing a host cell (or cell free expression system) containing polynucleotide (e.g. an expression vector) encoding the recombinant antigen binding polypeptide under conditions which permit expression of the antigen binding polypeptide, and recovering the expressed antigen binding polypeptide. This recombinant expression process can be used for large scale production of antigen binding polypeptides according to the invention, including monoclonal antibodies intended for human therapeutic use. Suitable vectors, cell lines and production processes for large scale manufacture of recombinant antibodies suitable for in vivo therapeutic use are generally available in the art and will be well known to the skilled person.

Further aspects of the invention relate to test kits, including diagnostic kits etc. comprising an antigen binding polypeptide according to the invention, and also pharmaceutical formulations comprising an antigen binding polypeptide according to the invention.

Where the antigen binding polypeptide is intended for diagnostic use, for example where the antigen binding polypeptide is specific for an antigen which is a biomarker of a disease state or a disease susceptibility, then it may be convenient to supply the antigen binding polypeptide as a component of a test kit. Diagnostic tests typically take the form of standard immunoassays, such as ELISA, radioimmunoassay, Elispot, etc. The components of such a test kit may vary depending on the nature of the test or assay it is intended to carry out using the antigen binding polypeptide of the invention, but will typically include additional reagents required to carry out an immunoassay using the antigen binding polypeptide of the invention. Antigen binding polypeptides for use as diagnostic reagents may carry a revealing label, such as for example a fluorescent moiety, enzymatic label, or radiolabel.

Antigen binding polypeptides intended for in vivo therapeutic use are typically formulated into pharmaceutical dosage forms, together with one or more pharmaceutically acceptable diluents, carriers or excipients (Remington's Pharmaceutical Sciences, 16th edition., Osol, A. Ed. 1980). Antigen binding polypeptides according to the invention are typically formulated as sterile aqueous solutions, to be administered intravenously, or by intramuscular, intraperitoneal, intra-cerebrospinal, intratumoral, oral, peritumoral, subcutaneous, intra-synovial, intrathecal, topical, sublingual or inhalation routes, to a mammalian subject, typically a human patient, in need thereof. For the prevention or treatment of disease, the appropriate dosage of antigen binding polypeptide will depend on the type of disease to be treated, the severity and clinical course of the disease, plus the patient's age, weight and clinical history, and will be determined by the judgement of the attending physician.

### Processes for the Production of Antigen Binding Polypeptides

A key aspect of the present invention relates to processes for the production of high affinity antigen binding polypeptides, and specifically monoclonal antibodies, against a target antigen of interest, wherein said target antigen is a polypeptide antigen that exhibits at least 90% amino acid sequence identity with a protein from said species in the family Camelidae.

Accordingly, the invention process for preparing a recombinant antigen binding polypeptide that specifically binds to a target antigen, said antigen binding polypeptide comprising a VH domain and a VL domain, wherein at least one hypervariable loop or complementarity determining region (CDR) in the VH domain or the VL domain is obtained from a species in the family Camelidae, said process comprising the steps of:

(a) immunising a species in the family Camelidae (with the target antigen), thereby raising a conventional antibody to said target antigen.

(b) isolating Camelidae nucleic acid encoding at least one hypervariable loop or complementarity determining region (CDR) of the VH and/or the VL domain of a Camelidae conventional antibody immunoreactive with said target antigen;

(c) preparing a polynucleotide comprising a nucleotide sequence encoding hypervariable loop(s) or complementarity determining region(s) having amino acid sequence identical to the hypervariable loop(s) or complementarity determining region(s) encoded by the nucleic acid isolated in step (a), which polynucleotide encodes an antigen binding polypeptide comprising a VH domain and a VL domain that is immunoreactive with (or specifically binds to) said target antigen; and

(d) expressing said antigen binding polypeptide from the recombinant polynucleotide of step (c), wherein said antigen binding polypeptide is not identical to the Camelidae conventional antibody of part (b), characterised in that said target antigen is a polypeptide antigen that exhibits at least 90%, or at least 95%, 96%, 97% or 98% amino acid sequence identity with a protein from said species in the family Camelidae.

The first step of this process involves active immunisation of a species in the family Camelidae in order to elicit an immune response against the target antigen, thereby raising camelid conventional antibodies immunoreactive with the target antigen. Protocols for immunisation of camelids are described in the accompanying examples. The antigenic material used for immunisation may be a purified form of the target antigen, for example recombinantly expressed polypeptide, or an immunogenic fragment thereof. However, it is also possible to immunise with crude preparations of the target antigen, such as like isolated cells or tissue preparations expressing or encoding the target antigen, cell lysates, cell supernatants or fractions such as cell membranes, etc., or lipoparticles, beads, vesicles or other particles containing the antigen on their surface, or with a polynucleotide encoding the target antigen (a DNA immunisation).

The process will typically involve immunisation of animals of a Camelidae species (including, but limited to, llamas and alpacas), and advantageously these animals will belong to a fully outbred population. However, it is also contemplated to use transgenic animals (e.g. transgenic mice) containing the Camelid conventional lg locus, or at least a portion thereof.

A key advantage of processes based on active immunisation of camelids stems from the fact that all species of Camelidae can be maintained in large outbred populations where the individual animals have a different genetic background. It is therefore possible to use active immunisation to elicit a strong and diverse immune response against the antigen of interest from which a diverse pool of potential antigen binding molecules can be obtained. As illustrated in the accompanying examples, active immunisation of camelids can generate Fab fragments binding to highly conserved target antigens (e.g. polypeptide antigens which share greater than 90% amino acid sequence identity with camelid polypeptides) with a high degree of immunodiversity.

Following active immunisation with the target antigen, peripheral blood lymphocytes or biopsies such as lymph nodes or spleen biopsies may be isolated from the immunised animal and screened for production of conventional camelid antibodies against the target antigen. Techniques such as enrichment using panning or FACS sorting may be used at this stage to reduce the complexity of the B cell repertoire to be screened, as illustrated in the examples. Antigen-specific B cells are then selected and used for total RNA extraction and subsequent cDNA synthesis. Nucleic acid encoding the native camelid VH and VL domains (specific for the target antigen) can be isolated by PCR.

Nucleic acid encoding camelid VH and VL domains may be cloned directly into an expression vector for the production of an antigen binding polypeptide according to the invention. In particular, these sequences could be cloned into an expression vector which also encodes a human antibody constant region, or a portion thereof, in order to produce a chimeric antibody. However, it is typical to carry out further manipulations on the isolated camelid VH and VL sequences before cloning and expression with human constant region sequences.

As a first step, candidate camelid VH and VL sequences (isolated following the active immunisation) may be used to prepare a camelid libraries (e.g. Fab libraries, as described in the accompanying examples). The library may then be screened (e.g. using phage display) for binding to the target antigen. Promising lead candidates can be further tested for target antigen binding, for example using Biacore or a suitable bioassay. Finally, the sequences encoding the VH and VL domains of the most promising leads can be cloned as an in-frame fusion with sequences encoding a human antibody constant region.

It is not essential that the polynucleotide sequence used to encode the (camelid-derived) HVs/CDRs (e.g. for recombinant expression of the antigen binding polypeptide of the invention) is identical to the native polynucleotide sequence which naturally encodes the HVs/CDRs in the camelid. Therefore, the invention encompasses/permits codon optimisation, and other changes in polynucleotide sequence related to cloning and/or expression, which do not alter the encoded amino acid sequence.

In certain embodiments, “chain shuffling” may be performed in which a particular variable domain known to bind the antigen of interest is paired with each of a set of variable domains of the opposite type (i.e. VH paired with VL library or vice versa), to create libraries, and the resulting combinations of VH/VL tested for antigen binding affinity and/or specificity. Alternatively, a library of VH domains could be paired with a library of VL domains, either randomly or in a hierarchical manner, and the resulting combinations tested (see Clackson et al., Nature., Vol. 352. pp624-638, 1991). In this process, the libraries may be libraries of rearranged VH and VL (Vκ or Vλ) from camelids which display immunity to the antigen of interest (including animals which have been actively immunised). The chain shuffling process can increase immunodiversity and produce pairings with significantly enhanced affinity.

In the processes of the invention, “native” camelid-derived VH and VL domains may be subject to protein engineering in which one or more selective amino acid substitutions are introduced, typically in the framework regions. The reasons for introducing such substitutions into the “wild type” camelid sequence can be (i) humanisation of the framework region, (ii) improvement in stability, bioavailability, product uniformity, tissue penetration, etc., or (iii) optimisation of target antigen binding.

“Germlining” of camelid-derived VH and VL domains by selective replacement of one or more amino acid residues in the framework regions may be carried out according to well-established principles (as illustrated in WO 2010/001251 and WO 2011/080350, the contents of which are specifically incorporated herein by reference). It will be appreciated that the precise identity of the amino acid changes made to achieve acceptable “germlining” of any given VH domain, VL domain or combination thereof will vary on a case-by-case basis, since this will depend upon the sequence of the framework regions derived from Camelidae and the starting homology between these framework regions and the closest aligning human germline (or somatically mutated) framework region, and possible also on the sequence and conformation of the hypervariable loops which form the antigen binding site. The overall aim of the germlining process is to produce a molecule in which the VH and VL domains exhibit minimal immunogenicity when introduced into a human subject, whilst retaining the specificity and affinity of the antigen binding site formed by the parental VH and VL domains encoded by Camelidae (e.g. camelid VH/VL obtained by active immunisation).

### Methods of Library Construction

In a related aspect, the invention also encompasses a process for producing a library of expression vectors encoding VH and VL domains of Camelidae conventional antibodies, said method comprising the steps:

a) actively immunising a camelid, thereby raising conventional camelid antibodies against a target antigen;

b) preparing cDNA or genomic DNA from a sample comprising lymphoid tissue (e.g. circulating B cells) from said immunised camelid;

c) amplifying regions of said cDNA or genomic DNA to obtain amplified gene segments, each gene segment comprising a sequence of nucleotides encoding a VH domain or a sequence of nucleotides encoding a VL domain of a Camelidae conventional antibody; and

d) cloning the gene segments obtained in c) into expression vectors, such that each expression vector contains a gene segment encoding a VH domain and a gene segment encoding a VL domain and directs expression of an antigen binding polypeptide comprising said VH domain and said VL domain, whereby a library of expression vectors is obtained, characterised in that said target antigen is a polypeptide antigen that exhibits at least 90%, or at least 95%, 96%, 97% or 98% amino acid sequence identity with a protein from said species in the family Camelidae.

The above methods of “library construction” may also form part of the general process for production of antigen binding polypeptides of the invention, described above. Hence, any feature described as being preferred or advantageous in relation to this aspect of the invention may also be taken as preferred or advantageous in relation to the general process, and vice versa, unless otherwise stated.

In one embodiment, the nucleic acid amplified in step a) comprises cDNA or genomic DNA prepared from lymphoid tissue of a camelid, said lymphoid tissue comprising one or more B cells, lymph nodes, spleen cells, bone marrow cells, or a combination thereof. Circulating B cells are particularly preferred. Peripheral blood lymphocytes (PBLs) can be used as a source of nucleic acid encoding VH and VL domains of conventional camelid antibodies, i.e. there is sufficient quantity of plasma cells (expressing antibodies) present in a sample of PBLs to enable direct amplification. This is advantageous because PBLs can be prepared from a whole blood sample taken from the animal (camelid). This avoids the need to use invasive procedures to obtain tissue biopsies (e.g. from spleen or lymph node), and means that the sampling procedure can be repeated as often as necessary, with minimal impact on the animal. For example, it is possible to actively immunise the camelid, remove a first blood sample from the animal and prepare PBLs, then immunise the same animal a second time, either with a “boosting” dose of the same antigen or with a different antigen, then remove a second blood sample and prepare PBLs.

Accordingly, a particular embodiment of this method may involve: preparing a sample containing PBLs from a camelid, preparing cDNA or genomic DNA from the PBLs and using this cDNA or genomic DNA as a template for amplification of gene segments encoding VH or VL domains of camelid conventional antibodies. In one embodiment the lymphoid tissue (e.g. circulating B cells) is obtained from a camelid which has been actively immunised, as described elsewhere herein. Conveniently, total RNA (or mRNA) can be prepared from the lymphoid tissue sample (e.g. peripheral blood cells or tissue biopsy) and converted to cDNA by standard techniques. It is also possible to use genomic DNA as a starting material.

This aspect of the invention encompasses both a diverse library approach, and a B cell selection approach for construction of the library. In a diverse library approach, repertoires of VH and VL-encoding gene segments may be amplified from nucleic acid prepared from lymphoid tissue without any prior selection of B cells. In a B cell selection approach, B cells displaying antibodies with desired antigen-binding characteristics may be selected, prior to nucleic acid extraction and amplification of VH and VL-encoding gene segments.

Various conventional methods may be used to select camelid B cells expressing antibodies with desired antigen-binding characteristics. For example, B cells can be stained for cell surface display of conventional IgG with fluorescently labelled monoclonal antibody (mAb, specifically recognizing conventional antibodies from llama or other camelids) and with target antigen labelled with another fluorescent dye. Individual double positive B cells may then be isolated by FACS, and total RNA (or genomic DNA) extracted from individual cells. Alternatively cells can be subjected to in vitro proliferation and culture supernatants with secreted IgG can be screened, and total RNA (or genomic DNA) extracted from positive cells. In a still further approach, individual B cells may be transformed with specific genes or fused with tumor cell lines to generate cell lines, which can be grown “at will”, and total RNA (or genomic DNA) subsequently prepared from these cells.

Instead of sorting by FACS, target specific B cells expressing conventional IgG can be “panned” on immobilized monoclonal antibodies (directed against camelid conventional antibodies) and subsequently on immobilized target antigen. RNA (or genomic DNA) can be extracted from pools of antigen specific B cells or these pools can be transformed and individual cells cloned out by limited dilution or FACS.

B cell selection methods may involve positive selection, or negative selection. Whether using a diverse library approach without any B cell selection, or a B cell selection approach, nucleic acid (cDNA or genomic DNA) prepared from the lymphoid tissue is subject to an amplification step in order to amplify gene segments encoding individual VH domains or VL domains.

Total RNA extracted from the lymphoid tissue (e.g. peripheral B cells or tissue biopsy) may be converted into random primed cDNA or oligo dT primer can be used for cDNA synthesis, alternatively Ig specific oligonucleotide primers can be applied for cDNA synthesis, or mRNA (i.e. poly A RNA) can be purified from total RNA with oligo dT cellulose prior to cDNA synthesis. Genomic DNA isolated from B cells can be used for PCR.

PCR amplification of heavy chain and light chain (kappa and lambda) gene segments encoding at least VH or VL can be performed with FR1 primers annealing to the 5′ end of the variable region in combination with primers annealing to the 3′ end of CH1 or Ckappa/Clambda region with the advantage that for these constant region primers only one primer is needed for each type. This approach enables camelid Fabs to be cloned. Alternatively sets of FR4 primers annealing to the 3′ end of the variable regions can be used, again for cloning as Fabs (fused to vector encoded constant regions) or as scFv (single chain Fv, in which the heavy and light chain variable regions are linked via a flexible linker sequence); alternatively the variable regions can be cloned in expression vectors allowing the production of full length IgG molecules displayed on mammalian cells.

In general the amplification is performed in two steps; in the first step with non-tagged primers using a large amount of cDNA (to maintain diversity) and in the second step the amplicons are re-amplified in only a few cycles with tagged primers, which are extended primers with restriction sites introduced at the 5′ for cloning. Amplicons produced in the first amplification step (non-tagged primers) may be gel-purified to remove excess primers, prior to the second amplification step. Alternatively, promoter sequences may be introduced, which allow transcription into RNA for ribosome display. Instead of restriction sites recombination sites can be introduced, like the Cre-Lox or TOPO sites, that permit the site directed insertion into appropriate vectors.

Amplified gene segments encoding camelid conventional VH and VL domains may then be cloned into vectors suitable for expression of VH/VL combinations as functional antigen binding polypeptides. By way of example, amplified VHCH1/VKCK/VLCL gene segments from pools of B cells (or other lymphoid tissue not subject to any B cell selection) may be first cloned separately as individual libraries (primary libraries), then in a second step Fab or scFV libraries may be assembled by cutting out the light chain fragments and ligating these into vectors encoding the heavy chain fragments. The two step procedure supports the generation of large libraries, because the cloning of PCR products is relatively inefficient (due to suboptimal digestion with restriction enzymes). scFv encoding DNA fragments can be generated by splicing-by-overlap extension PCR (SOE) based on a small overlap in sequence in amplicons; by mixing VH and VL encoding amplicons with a small DNA fragment encoding the linker in a PCR a single DNA fragment is formed due to the overlapping sequences.

Amplicons comprising VH and VL-encoding gene segments can be cloned in phage or phagemid vectors, allowing selection of target specific antibody fragments by using phage display based selection methods. Alternatively amplicons can be cloned into expression vectors which permit display on yeast cells (as Fab, scFv or full length IgG) or mammalian cells (as IgG).

In other embodiments, cloning can be avoided by using the amplicons for ribosome display, in which a T7 (or other) promoter sequence and ribosome binding site is included in the primers for amplification. After selection for binding to target antigen, pools are cloned and individual clones are analyzed. In theory, larger immune repertoires can be sampled using this approach as opposed to a phage display library approach, because cloning of libraries and selection with phage is limited to 10¹⁰ to 10¹² clones.

When applying B cell sorting, amplicons contain VH or VL-encoding gene segments of individual target specific B cells can be cloned directly into bacterial or mammalian expression vectors for the production of antibody fragments (scFVs or Fabs) or even full length IgG.

In a particular, non-limiting, embodiment of the “library construction” process, the invention provides a method of producing a library of expression vectors encoding VH and VL domains of camelid conventional antibodies, said method comprising the steps:

a) actively immunising a camelid, thereby raising conventional camelid antibodies against a target antigen;

b) preparing cDNA or genomic DNA from a sample comprising lymphoid tissue (e.g. circulating B cells) from said immunised camelid (including, but not limited to, Llama or alpaca);

c) amplifying regions of said cDNA or genomic DNA to obtain amplified gene segments, each gene segment comprising a sequence of nucleotides encoding a VH domain or a sequence of nucleotides encoding a VL domain of a camelid conventional antibody; and

d) cloning the gene segments obtained in c) into expression vectors, such that each expression vector contains a gene segment encoding a VH domain and a gene segment encoding a VL domain and directs expression of an antigen binding polypeptide comprising said VH domain and said VL domain, whereby a library of expression vectors is obtained characterised in that the antigen binding polypeptide specifically binds to a target antigen which is a polypeptide antigen that exhibits at least 90%, or at least 95%, 96%, 97% or 98% amino acid sequence identity with a protein from said species in the family Camelidae.

The foregoing methods may be used to prepare libraries of camelid-encoded VH and VL domains (in particular Llama and alpaca VH and VL domains), suitable for expression of VH/VL combinations as functional antigen-binding polypeptides, e.g. in the form of scFVs, Fabs or full-length antibodies.

Libraries of expression vectors prepared according to the foregoing process, and encoding camelid (including but not limited to Llama or alpaca) VH and VL domains, also form part of the subject-matter of the present invention.

In a particular embodiment the invention provides a library of phage vectors encoding Fab or scFV molecules, wherein each Fab or scFV encoded in the library comprises a VH domain of a camelid conventional antibody and a VL domain of a camelid conventional antibody.

In one embodiment the library is a “diverse” library, in which the majority of clones in the library encode VH domains of unique amino acid sequence, and/or VL domains of unique amino acid sequence, including diverse libraries of camelid VH domains and camelid VL domains. Therefore, the majority (e.g. >90%) of clones in a diverse library encode a VH/VL pairing which differs from any other VH/VL pairing encoded in the same library with respect to amino acid sequence of the VH domain and/or the VL domain.

The invention also encompasses expression vectors containing VH and VL-encoding gene segments isolated from a single selected B cell of a camelid (e.g. Llama or alpaca).

In a further aspect, the present invention also provides a method of selecting an expression vector encoding an antigen binding polypeptide immunoreactive with a target antigen, the method comprising steps of:

i) providing a library of expression vectors, wherein each vector in said library comprises a gene segment encoding a VH domain and a gene segment encoding a VL domain, wherein at least one of said VH domain or said VL domain is from a camelid conventional antibody, and wherein each vector in said library directs expression of an antigen binding polypeptide comprising said VH domain and VL domain;

ii) screening antigen binding polypeptides encoded by said library for immunoreactivity with said target antigen, and thereby selecting an expression vector encoding an antigen binding polypeptide immunoreactive with said target antigen, characterised in that the antigen binding polypeptide specifically binds to a target antigen which is a polypeptide antigen that exhibits at least 90%, or at least 95%, 96%, 97% or 98% amino acid sequence identity with a protein from said species in the family Camelidae.

This method of the invention encompasses screening/selection of clones immunoreactive with target antigen, which target antigen is a polypeptide antigen that exhibits at least 90% amino acid sequence identity with a protein from said species in the family Camelidae, from a library of clones encoding VH/VL pairings. The method may also encompass library construction, which may be carried out using the library construction method described above. Optional downstream processing/optimisation steps may be carried out on selected clones, as described below. This method of selection and screening may also form part of the general process for production of antigen binding polypeptides of the invention, described above. Hence, any feature described as being preferred or advantageous in relation to this aspect of the invention may also be taken as preferred or advantageous in relation to the general process, and vice versa, unless otherwise stated.

Screening and Selection of Clones Immunoreactive with Target Antigen

Screening/selection typically involves contacting expression products encoded by clones in the library (ie. VH/VL pairings in the form of antigen binding polypeptides, e.g. Fabs, scFVs or antibodies) with a target antigen, and selecting one or more clones which encode a VH/VL pairings exhibiting the desired antigen binding characteristics, i.e. binding to a target antigen which is a polypeptide antigen that exhibits at least 90% amino acid sequence identity with a protein from said species in the family Camelidae.

Phage display libraries may be selected on immobilized target antigen or on soluble (often biotinylated) target antigen. The Fab format allows affinity driven selection due to its monomeric appearance and its monovalent display on phage, which is not possible for scFv (as a consequence of aggregation and multivalent display on phage) and IgG (bivalent format). Two to three rounds of selections are typically needed to get sufficient enrichment of target specific binders.

Affinity driven selections can be performed by lowering the amount of target antigen in subsequent rounds of selection, whereas extended washes with non-biotinylated target enables the identification of binders with extremely good affinities.

The selection procedure allows the user to home in on certain epitopes; whereas the classical method for elution of phage clones from the immobilized target is based on a pH shock, which denatures the antibody fragment and/or target, competition with a reference mAb against the target antigen or soluble receptor or cytokine leads to the elution of phage displaying antibody fragments binding to the relevant epitope of the target (this is of course applicable to other display systems as well, including the B cells selection method).

Individual clones taken from the selection outputs may be used for small scale production of antigen-binding polypeptides (e.g. antibody fragments) using periplasmic fractions prepared from the cells or the culture supernatants, into which the fragments “leaked” from the cells. Expression may be driven by an inducible promoter (e.g. the lac promoter), meaning that upon addition of the inducer (IPTG) production of the fragment is initiated. A leader sequence ensures the transport of the fragment into the periplasm, where it is properly folded and the intramolecular disulphide bridges are formed.

The resulting crude protein fractions may be used in target binding assays, such as ELISA. For binding studies, phage prepared from individual clones can be used to circumvent the low expression yields of Fabs, which in general give very low binding signals. These protein fractions can also be screened using in vitro receptor—ligand binding assays to identify antagonistic antibodies; ELISA based receptor—ligand binding assays can be used, also high throughput assays like Alphascreen are possible. Screening may be performed in radiolabelled ligand binding assays, in which membrane fractions of receptor overexpressing cell lines are immobilized; the latter assay is extremely sensitive, since only picomolar amounts of radioactive cytokine are needed, meaning that minute amounts of antagonistic Fabs present in the crude protein fraction will give a positive read-out. Alternatively, FACS can be applied to screen for antibodies, which inhibit binding of a fluorescently labelled cytokine to its receptor as expressed on cells, while FMAT is the high throughput variant of this.

Fabs present in periplasmic fractions or partially purified by IMAC on its hexahistidine tag or by protein G (known to bind to the CH1 domain of Fabs) can be directly used in bioassays using cells, which are not sensitive to bacterial impurities; alternatively, Fabs from individual E. coli cells can be recloned in mammalian systems for the expression of Fabs or IgG and subsequently screened in bioassays.

Following identification of positive expression vector clones, i.e. clones encoding a functional VH/VL combination which binds to the desired target antigen, it is a matter of routine to determine the nucleotide sequences of the variable regions, and hence deduce the amino acid sequences of the encoded VH and VL domains.

If desired, the Fab (or scFV) encoding region may be recloned into an alternative expression platform, e.g. a bacterial expression vector (identical to the phagemid vector, but without the gene 3 necessary for display on phage), which allows larger amounts of the encoded fragment to be produced and purified.

The affinity of target binding may be determined for the purified Fab (or scFV) by surface plasmon resonance (e.g. Biacore) or via other methods, and the neutralizing potency tested using in vitro receptor—ligand binding assays and cell based assays.

Families of antigen-binding, and especially antagonistic Fabs (or scFVs) may be identified on the basis of sequence analysis (mainly of VH, in particular the length and amino acid sequence of CDR3 of the VH domain).

### Potency Optimisation

Clones identified by screening/selection as encoding a VH/VL combination with affinity for the desired target antigen may, if desired, be subject to downstream steps in which the affinity and/or neutralising potency is optimised.

Potency optimization of the best performing member of each VH family can be achieved via light chain shuffling, heavy chain shuffling or a combination thereof, thereby selecting the affinity variants naturally occurring in the animal. This is particularly advantageous in embodiments where the original camelid VH/VL domains were selected from an actively immunised camelid, since it is possible to perform chain shuffling using the original library prepared from the same immunised animal, thereby screening affinity variants arising in the same immunised animal.

For light chain shuffling the gene segment encoding the VH region (or VHCH1) of VH/VL pairing with desirable antigen binding characteristics (e.g. an antagonistic Fab) may be used to construct a library in which this single VH-encoding gene segment is combined with the light chain repertoire of the library from which the clone was originally selected. For example, if the VH-encoding segment was selected from a library (e.g. Fab library) prepared from a camelid animal actively immunised to elicit an immune response against a target antigen, then the “chain shuffling” library may be constructed by combining this VH-encoding segment with the light chain (VL) repertoire of the same immunised camelid. The resulting library may then be subject to selection of the target antigen, but under stringent conditions (low concentrations of target, extensive washing with non-biotinylated target in solution) to ensure the isolation of the best affinity variant. Off-rate screening of periplasmic fractions may also assist in the identification of improved clones. After sequence analysis and recloning into a bacterial production vector, purified selected Fabs may be tested for affinity (e.g. by surface plasmon resonance) and potency (e.g. by bioassay).

Heavy chain shuffling can be performed by cloning back the gene segment encoding the light chain (VL) of a clone selected after light-chain shuffling into the original heavy chain library from the same animal (from which the original VH/VL-encoding clone was selected). Alternatively a CDR3 specific oligonucleotide primer can be used for the amplification of the family of VH regions, which can be cloned as a repertoire in combination with the light chain of the antagonistic Fab. Affinity driven selections and off-rate screening then allow the identification of the best performing VH within the family.

It will be appreciated that the light chain shuffling and heavy chain shuffling steps may, in practice, be performed in either order, i.e. light chain shuffling may be performed first and followed by heavy chain shuffling, or heavy chain shuffling may be performed first and followed by light chain shuffling. Both possibilities are encompassed within the scope of the invention. In other circumstances it may not be necessary to perform both light chain shuffling and heavy chain shuffling, so processes involving only light chain shuffling or only heavy chain shuffling are also encompassed.

From light chains or heavy chains of VH/VL pairings (e.g. Fabs) with improved affinity and potency the sequences of, in particular, the CDRs can be used to generate engineered variants in which mutations of the individual Fabs are combined. It is known that often mutations can be additive, meaning that combining these mutations may lead to an even more increased affinity.

### Germlining and Formatting for Human Therapeutic Use

The VH and VL-encoding gene segments of selected expression clones encoding VH/VL pairings exhibiting desirable antigen-binding characteristics (e.g. phage clones encoding scFVs or Fabs) may be subjected to downstream processing steps and recloned into alternative expression platforms, such as vectors encoding antigen binding polypeptide formats suitable for human therapeutic use (e.g. full length antibodies with fully human constant domains).

Promising “lead” selected clones may be engineered to introduce one or more changes in the nucleotide sequence encoding the VH domain and/or the VL domain, which changes may or may not alter the encoded amino acid sequence of the VH domain and/or the VL domain. Such changes in sequence of the VH or VL domain may be engineered for any of the purposes described elsewhere herein, including germlining or humanisation, codon optimisation, enhanced stability, optimal affinity etc.

The general principles of germlining described herein apply equally in this embodiment of the invention. By way of example, lead selected clones containing camelid-encoded VH and VL domains may be germlined in their framework regions (FRs) by applying a library approach. After alignment against the closest human germline (for VH and VL) and other human germlines with the identical canonical folds of CDR1 and CDR2, the residues to be changed in the FRs are identified and the preferred human residue selected, as described in WO 2010/001251 and WO 2011/080350. Whilst germlining may involve replacement of camelid-encoded residues with an equivalent residue from the closest matching human germline this is not essential, and residues from other human germlines could also be used.

Once the amino acid sequences of the lead VH and VL domains (following potency optimisation, as appropriate) are known, synthetic genes of VH and VL can be designed, in which residues deviating from the human germline are replaced with the preferred human residue (from the closest matching human germline, or with residues occurring in other human germlines, or even the camelid wild type residue). At this stage the gene segments encoding the variable domains may be re-cloned into expression vectors in which they are fused to human constant regions of the Fab, either during gene synthesis or by cloning in an appropriate display vector.

The resulting VH and VL synthetic genes can be recombined into a Fab library or the germlined VH can be recombined with the wild type VL (and vice versa, referred to as “hybrid” libraries). Affinity-driven selections will allow the isolation of the best performing germlined version, in case of the “hybrid” libraries, the best performing germlined VH can be recombined with the best performing germlined VL.

Amino acid and nucleotide sequence information for the germlined Fabs can be used to generate codon-optimized synthetic genes for the production of full length human IgG of the preferred isotype (IgG1 for ADCC and CDC, IgG2 for limited effector functions, IgG4 as for IgG2, but when monovalent binding is required). For non-chronic applications and acute indications bacterially or mammalian cell produced human Fab can produced as well.

Combining steps of the above-described processes, in a particular non-limiting embodiment the present invention provides a method of producing an expression vector encoding a chimeric antigen binding polypeptide immunoreactive with a target antigen, said method comprising the steps of:

a) actively immunising a camelid (including but not limited to Llama or alpaca), thereby raising conventional camelid antibodies against a target antigen;

b) preparing cDNA or genomic DNA from a sample comprising lymphoid tissue (e.g. circulating B cells) from said immunised camelid;

c) amplifying regions of said cDNA or genomic DNA to obtain amplified gene segments, each gene segment comprising a sequence of nucleotides encoding a VH domain or a sequence of nucleotides encoding a VL domain of a camelid conventional antibody;

d) cloning the gene segments obtained in c) into expression vectors, such that each expression vector contains a gene segment encoding a VH domain and a gene segment encoding a VL domain and directs expression of an antigen binding polypeptide comprising said VH domain and said VL domain, thereby producing a library of expression vectors;

e) screening antigen binding polypeptides encoded by the library obtained in step d) for immunoreactivity with said target antigen, and thereby selecting an expression vector encoding an antigen binding polypeptide immunoreactive with said target antigen;

f) optionally performing a light chain shuffling step and/or a heavy chain shuffling step to select an expression vector encoding a potency-optimised antigen binding polypeptide immunoreactive with said target antigen;

g) optionally subjecting the gene segment encoding the VH domain of the vector selected in step e) or step f) and/or the gene segment encoding the VL domain of the vector selected in step e) or step f) to germlining and/or codon optimisation; and

h) cloning the gene segment encoding the VH domain of the vector selected in part e) or f) or the germlined and/or codon optimised VH gene segment produced in step g) and the gene segment encoding the VL domain of the vector selected in part e) or f) or the germlined and/or codon optimised VL gene segment produced in step g) into a further expression vector, in operable linkage with a sequence of nucleotides encoding one or more constant domains of a human antibody, thereby producing an expression vector encoding a chimeric antigen binding polypeptide comprising the VH and VL domains fused to one or more constant domains of a human antibody, characterised in that the antigen binding polypeptide specifically binds to a target antigen which is a polypeptide antigen that exhibits at least 90%, or at least 95%, 96%, 97% or 98% amino acid sequence identity with a protein from said species in the family Camelidae.

The invention also extends to expression vectors prepared according to the above-described processes, and to a method of producing an antigen binding polypeptide immunoreactive with a target antigen, the method comprising steps of:

a) preparing expression vector encoding an antigen binding polypeptide immunoreactive with a target antigen using the method described above;

b) introducing said expression vector into host cell or cell-free expression system under conditions which permit expression of the encoded antigen binding polypeptide; and

c) recovering the expressed antigen binding polypeptide, again characterised in that the antigen binding polypeptide specifically binds to a target antigen which is a polypeptide antigen that exhibits at least 90%, or at least 95%, 96%, 97% or 98% amino acid sequence identity with a protein from said species in the family Camelidae.

In one embodiment, the latter process encompasses bulk production-scale manufacture of the antigen-binding polypeptide of the invention, particularly bulk-scale manufacture of therapeutic antibodies intended for use as pharmaceutically active agents, by recombinant expression. In such embodiments, the expression vector prepared in step a) and the host cell/expression system used in step b) are selected to be suitable for large-scale production of recombinant antibodies intended for administration to human patients. The general characteristics of suitable vectors and expression systems for this purpose are well known in the art.

The invention will be further understood by reference to the following non-limiting experimental examples.

### EXPERIMENTAL EXAMPLES

### Example 1

### Antibodies to HMGB1

### 1.1 Target Antigen

The target antigen for this example was high mobility group box 1 (HMGB1), a highly conserved, ubiquitous protein present in the nuclei and cytoplasm of nearly all cell types. The human and mouse HMGB1 are 215 aa in length and 97% identical in sequence. HMGB1 is a non-histone chromatin associated protein and a regulator of gene function, but it also acts as an alarmin.

HMGB1 has been linked with inflammatory conditions, such as sepsis, SLE and RA, and also various cancers.

One of the reasons to address this target is that it is an extremely conserved target and difficult to raise antibodies by active immunization, because of the removal of anti-self antibodies by the immune system. HMGB1 is highly conserved, as illustrated by the fact that there is 97% identity in amino acid sequence between the human and mouse/rat proteins. To assess the degree of conservation between the human and llama HMGB1 homologs BLAST searches were performed with human HMGB1 sequence on the Whole Genome Shotgun sequence database from Lama pacos (www.ncbi.nlm.nih.gov/nuccore/206581138). These searches identified three genomic sequences, of which one contains the first two exons (sequence ID: ABRR01273630.1) and the other encodes the fourth and last exon (sequence ID: ABRR01273631.1), while the central part of the gene is encoded by another hit (sequence ID: ABRR01227803.1) indicating that the organization of the llama HMGB1 gene is identical to the organization of the human gene. The overall sequence identity between human and llama HMGB1 is 96%, although it has to be emphasized that the sequences from the Whole Genome Shotgun database are not completely reliable and may contain sequencing errors.

### 1.2 Immunisation of Llamas

Four llamas were immunized with recombinant HMGB1 (HMGBiotech, HM114), using Freund's incomplete adjuvant. During the first injection 80 μg of target protein was given, followed by four injections of 40 μg of target protein administered every two weeks. Three days after the last injection 400 ml blood was sampled for purification of PBLs.

### 1.3 Construction of Fab Libraries

PBLs isolated from the four llamas immunized with the HMGB1 antigen in Freund's incomplete adjuvant were used for RNA extraction, RT-PCR and PCR-cloning of Fab in a phagemid (pCB3) using the two-step cloning strategy described by De Haard H, et al., J. Biol. Chem. 274, 1999. For amplification of the antibody repertoires the amplification primers described in WO 2010/001251 were applied.

Independent VλCλ and VκCκ (libraries were constructed using a two step PCR, in which 25 cycles with non-tagged primers was done followed by 10 cycles with the tagged version of these primers (containing restriction sites for repertoire cloning). The VHCH1 libraries were built in parallel using the same approach. The sets of primers used for the amplification of the VH, Vλ and Vκ genes can be found in WO 2010/001251. The primary heavy and light chain libraries were made in pCB3, the sizes of these libraries ranged from 1×10⁷ to 3×10⁸ colonies.

Next, the light chain encoding inserts from the primary VλCλ and VκCκ libraries were re-cloned separately in the VHCH1-expressing vector to create the “Lambda” and “Kappa” Fab-library, respectively. Quality control of the libraries routinely performed by PCR gave insight about the percentage of clones with full length Fab insert. The Fab libraries obtained were large, having sizes of 2-9×10⁸ colonies. A high number of clones tested randomly from each library contained full length Fab sequences, indicating the high quality of the libraries.

### 1.4 Selection of HMGB1 Specific Fabs

Phage display was used to recruit a diverse panel of llama Fabs binding to directly coated HMGB1. After four rounds of selection good enrichments were obtained upon elution with trypsin.

Individual clones, originating from these selections, were grown in a 96-deep well plate (1 ml expressions) and phages were prepared and tested in a phage binding ELISA. In brief, phages derived from individual clones were incubated on a HMGB1 (100 ng/100 μl, Genway (10-663-46237)) and casein-coated well to assay specific binding to HMGB1. Phage binding was detected using an anti-M13-HRP antibody (GE Healthcare, 27-9421-01).

To test sequence diversity of the clones that bound specifically to HMGB1 a fingerprint analysis was performed. Therefore the Fab-encoding inserts of these clones were amplified by standard PCR techniques. The PCR fragments were digested with restriction enzymes Haelll and Alul and the digested samples were run on a 2.5% agarose gel. Some clones/fingerprints were represented multiple times (results not shown), but overall, diversity was high.

Clones with a unique fingerprint were again grown in a 96-deep well plate (1 ml expressions) and both phages and periplasmic extracts were prepared, which were respectively re-tested in a phage ELISA and a Fab ELISA for HMGB1 binding (data not shown). In both cases, most clones again scored positive for HMGB1 binding.

Sequences of both VH and Vλ or Vκ fragments were determined for each clone. A total of 6 different VH families were obtained based on the HCDR3 sequence, comprising VH and Vλ affinity variants, i.e. containing somatic mutations. Both clones containing Vλ and Vκ light chains were found.

TABLE 1
Closest human germline and % human identity and % human
homology for all HMGB1 binding clones.

| | | VH | | | VL | | |
|---|---|---|---|---|---|---|---|
| | | Closest | | | Closest | | % |
| | Name | human | % | % | human | % | homol- |
| Family | clone | germline | identity | homology | germline | identity | ogy |
| 1 | 3A1 | VH4 | 72% | 75% | VL1 | 84% | 89% |
| 2 | 2C1 | VH4 | 84% | 85% | VL3 | 86% | 91% |
| 2 | 3A4 | VH4 | 85% | 86% | VL3 | 86% | 91% |
| 3 | 3G1 | VH4 | 86% | 87% | VL5 | 90% | 94% |
| 4 | 2A1 | VH3 | 86% | 92% | VL3 | 77% | 78% |
| 5 | 2D6 | VH3 | 90% | 93% | VK4 | 83% | 83% |
| 6 | 3F11 | VH3 | 90% | 94% | VK2 | 85% | 89% |
| 6 | 3A12 | VH3 | 90% | 94% | VK2 | 86% | 89% |
| 6 | 2B3 | VH3 | 90% | 94% | VK2 | 86% | 89% |

### 1.5 Off-Rate and HMGB1 Specificity

Periplasmic extracts were prepared at a 20 ml scale for all HMGB1 binding clones with unique sequences. These periplasmic extracts were tested for off rate in Biacore on a HMGB1 coated CM5 chip (see Table 2, second column).

Large scale expressions (350 ml scale) were initiated for selected clones, from which Fabs were purified by IMAC using TALON beads. The purified Fabs were again tested for off rate in Biacore (third column in Table 2). HMGB2, which shares 81% sequence homology with HMGB1, was coated on the same chip. Off rate values for HMGB2 for both periplasmic extracts and purified Fabs are also shown in Table 2.

TABLE 2
Off rate values as determined in Biacore on a CM5
chip coated with HMGB1/HMGB2 for periplasmic extracts
(coded peri) and purified (pur) Fabs.

| | |
|---|---|
| HMGB1 | HMGB2 |

| | k_{off} peri | k_{off} pur | k_{off} peri | k_{off} pur |
|---|---|---|---|---|
| Clone | (10⁻⁴ s⁻¹) | (10⁻⁴ s⁻¹) | (10⁻⁴ s⁻¹) | (10⁻⁴ s⁻¹) |
| 3A1 | 5.93 | | 12 | |
| 2C1 | 11.9 | | 8.31 | |
| 2D2 | 5.59 | | 3.58 | |
| 3A4 | 3.44 | 7.51 | 4.77 | 2.57 |
| 3G1 | 176 | | 16.5 | |
| 2A1 | 5.79 | 2.36 | No binding | No binding |
| 2D6 | nd | 2.04 | nd | 2.89 |
| 3A12 | 8.13 | | 7.39 | |
| 2B3 | 13.4 | | 9.73 | |
| 3F11 | 8.16 | | 13.2 | 4.41 |

### 1.6 Reactivity Against HMGB1 and HMGB2

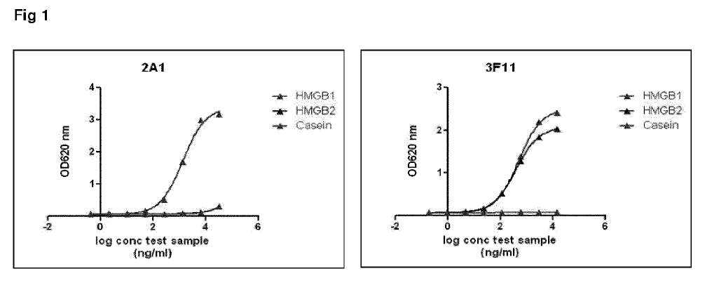

Purified Fabs of clone 2A1 and 3F11 were tested in a binding ELISA to confirm the different HMGB1/HMGB2 binding properties observed by Biacore (see Table 2). Therefore, HMGB1, HMGB2 or casein was coated directly on a microtiter plate. Fab binding was detected using an anti-myc-HRP antibody (Imtec Diagnostics, A190-105P). Results are shown in FIG. 1. The ELISA data confirm the HMGB2 cross-reactivity profile of SIMPLE antibody 3F11 and the lack of cross-reactivity of SIMPLE antibody 2A1 as was observed in Biacore.

### 1.7 Antagonistic Properties of Anti-HMGB1 Antibodies

Purified Fabs 2A1 and 3F11 were tested for their ability to compete for binding of HMGB1 to its ligand RAGE. Therefore, an ELISA based binding assay was developed in which HMGB1 was coated and RAGE-Fc fusion (R&D systems, 1145-RG) was allowed to interact with the target. Bound RAGE was detected with an HRP conjugated anti-Fc antibody (Jackson ImmunoResearch, 109-035-008).

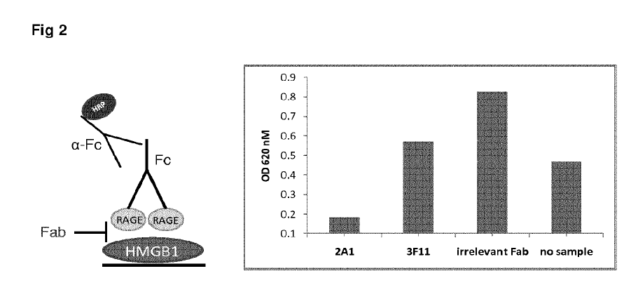

Fabs of clone 2A1 and 3F11 and an irrelevant Fab (5 μg/ml) were incubated in the wells coated with HMGB1 one hour prior to the addition of RAGE-Fc (final concentration 200 ng/ml). As can be seen in FIG. 2 the HMGB1 specific Fab 2A1 gave a much lower signal than when no sample or the irrelevant Fab was added. In contrast, the HMGB2 cross-reactive Fab3F11 gives a signal comparable to the control wells where no sample was added.

It can be concluded that Fab 2A1 antagonizes the binding of HMGB1 to RAGE, whereas Fab 3F11 does not affect this interaction.

### Example 2

### Anti-idiotype Antibodies to Llama Fabs Binding a Human Cytokine Antigen

### 2.1 Target Antigens

The target antigens for this example were two llama-derived monoclonal antibodies which specifically bind to a human target protein which is a cytokine. These monoclonal antibodies comprise llama-derived Fab regions (denoted 129D3/68F2 and 111A7/61H7) formatted with the constant regions (Fc) of a human antibody.

Fab 129D3 is a germlined variant of a llama-originating Fab denoted 68F2. The llama-derived Fab 68F2 was itself raised by immunisation of a fully outbred llama with the target antigen (a human cytokine). Fab 129D3 is a variant derived from 68F2 by the introduction of 13 amino acid substitutions within the framework regions, in order to increase the overall sequence identity up to 95.2% with the closest human germline. 129D3 and 68F2 exhibit the same binding specificity for the target human antigen, however Fab 129D3 is more suitable for use as a human therapeutic agent due to its higher human FR sequence identity, particularly when formatted as a monoclonal antibody with a fully human Fc region. The total amino acid sequence identity between 129D3 and 68F2 (across both VH and VL) is 94%.

The second llama-derived Fab 111A7 is a germlined variant of a llama-originating Fab denoted 61 H7. Fab 61 H7 was also raised by immunisation of a fully outbred llama with the target antigen (a human cytokine). Fab 111A7 is a variant derived from 61 H7 by the introduction of 13 amino acid substitutions within the framework regions, in order to increase the overall sequence identity up to 96.3% with the closest human germline. 11 1A7 and 61 H7 exhibit the same binding specificity and affinity/potency for the target human antigen. The total amino acid sequence identity between 111A7 and 61 H7 (across both VH and VL) is 93%.

Fab 103A1 is a sequence variant of 61 H7 which is specific for the same human cytokine as 61H7.

There is a commercial interest in developing anti-idiotype Fabs (or mAbs) which bind specifically to Fab 129D3 (i.e. the germlined variant of 68F2) and Fab 111A7 (i.e. the germlined variant of 61 H7) particularly to facilitate pharmacokinetic (PK) studies required during clinical trials of mAbs containing Fab 129D3 or Fab 111A7.

### 2.2 Immunization of Llamas

In order to optimise production of anti-idiotypic antibodies against monoclonal antibodies 68F2 and 61 H7, the antibodies were first engineered to replace the human constant regions with the constant regions of llama IgG1, ie conventional antibody type in llamas. This technical feature drives the immune response towards V regions and not against the llama Fc, ensuring that the immune response of the llama is focused against the specific CDRs (the idiotype) of 68F2 and 61 H7 and not against the constant regions of the antibodies. In addition, monoclonal antibodies containing the Fc of llama IgG1 will exhibit a long half-life in the llama following immunisation, meaning the immunogen is around for a long time helping the immune system to mount a response. Sequences of llama IgG1 (heavy chain) and llama CKappa and CLambda are given in PCT publication WO 2011/001251.

For active immunization with the target monoclonal antibodies two llamas were used. Llamas received antigen (target mAbs) by injecting intramuscularly in the neck during 6 weeks on once-a-week basis. Antigen was aliquoted in 500 μl fractions for the weekly injection for a single llama. 500 μl contained 100 μg antigen for the first two weeks. The remaining four weeks each injection contained 50 μg antigen/500 μl. The antigen was buffered in PBS (phosphate buffered saline). Antigen consisted of mAbs L68F2, L61 H7 and L103A1 in a ratio of 2:1:1. Before injection the antigen was mixed with Incomplete Freund's Adjuvant (IFA). IFA consists of paraffin and mannide mono-oleate. It enhances the lifetime of antigens and enhances transport to critical sites of the immune system. This amplifies the immune response.

On day zero serum was collected from the llama and on day 40 (five days after last immunization) 400 ml immune blood containing peripheral blood lymphocytes (PBLs) was collected. Peripheral blood lymphocytes were purified by centrifuging on a Ficoll-Paque gradient and used for extraction of total RNA.

### 2.3 Library Construction

Total RNA was converted into random primed cDNA using reverse transcriptase and gene sequences encoding for the heavy chain (VHCH1) and the two types of light chains (VκCκ and VλCλ) were amplified by PCR using this cDNA as template. Using a few intermediate cloning steps, combinatorial Fab libraries were made in a ρCB3 vector in which all different VHCH1 amplicons were combined with all different light chain amplicons. This was performed for both the κ-type light chains and the λ-type light chains. Consequently, this resulted for each llama in 2 Fab libraries: a κ-library and a λ-library.

The preferred size of the library was >1×10⁹ CFUs with a correct-insert ratio above 75% to cover the complete antibody repertoire as comprehensively as possible and raising the chance that the original VH-VL combinations are present.

### 2.4 Selections and Screening

The Fab libraries (plasmids) were transformed in bacteria which subsequently were infected with VCSM13 helper phage. As a result the E coli cells produced and secreted phages which displayed a single copy of the Fab fragment encoded by the phagemid genome present in the infected bacterium. The phages were purified from the bacteria using the PEG precipitation method. These phages were used for selections.

### Selection Method 1: a Method Based on Counter Selection Using Human Serum:

The counter selection against human serum is intended to remove Fabs from the library that recognize common epitopes on human antibodies. Phage prepared from the library and purified by PEG precipitation was diluted tenfold in 20% normal human serum (containing approximately 3 mg/ml IgG1)/PBS and incubated 30 minutes in a head-over-head rotator prior to transfer of 100 μl of this mixture to wells coated with L68F2 or L61H7.

Selection method 1 was performed on κ and λ libraries from one of the immunised llamas and resulted in the identification of five Fabs against 61 H7 (103A1): 8D6/8H7 from the κ-library and 7C6/7B4/7C12 from the λ-library. These five Fabs were re-cloned into a special expression vector (pCB4) to produce proper amounts of Fab protein for further characterization. In addition, the variable domains of the Fabs 8H7 and 7C6 were fused with the constant domains of llama and human IgG1.

### Selection Method 2: a Method Based on Counter Selection Using Human Serum and a Non-Relevant Isotypic Human Monoclonal Antibody (36C4):

Again the counter selection is intended to remove Fabs from the library that recognize common epitopes on human antibodies. Phage was diluted tenfold in 20% normal human serum, but now a human mAb was added at a concentration of 500 μg/ml.

Selection method 2 resulted in the identification of one Fab, 11E1, against 68F2. The heavy chain and light chain variable domains of the 11 E1 Fab were fused to the heavy chain and light chain constant domains of human and llama IgG1. Both the human IgG1 format, named hu11E1, and the llama IgG1 format, named la11E1, were expressed in HEK293 cells and provided sufficient protein for characterization.

### 2.5 Characterisation of Anti-Idiotypic Antibodies

2.5.1 Characterization of Anti-Idiotypic Antibody 11E1 against 68F2

Once anti-idiotypic human and llama IgG versions of 11E1 were available different experiments were performed to characterize the molecule better:

Surface plasmon resonance analysis to define the affinity and the off rate of hu11E1 (human IgG version of 11E1)

ELISAs to determine the binding profile of la11E1 (llama IgG version of 11E1) against different mAbs in order to determine the specificity of the molecule

Pharmacokinetic experiments to prove that la11E1 can be used as a reliable tool for pharmacokinetic studies

2.5.1.1: Determination of Dissociation Constant (K_{D}) and Off Rate (k_{off}) of the Interaction Between Anti-Idiotypic mAb hu11E1 and Idiotypic mAb 68F2.

Binding of anti-idiotypic Fabs specific for 68F2 extracted from the periplasmic fraction were screened on binding to the idiotype using SPR. This was done using a Biacore 3000 (Biacore AB/GE Healthcare). 68F2 served as ligand and was immobilized in flowcell 2 of a CM5 chip (Biacore AB) with a density of 3000 response units [RUs]. As background control PBS was ‘immobilized’ in flowcell 1. Periplasmic fractions were diluted with a factor 8 in running buffer HBS-EP (10 mM HEPES, 3 mM EDTA, 150 mM NaCl, 0.005% Tween-20). The flow was set at 30 μl/min and analyte injection consisted of 60 μl analyte. Furthermore a dissociation time of 3 minutes was set and kinetics were measured. All used procedures were performed according to recommendations of the manufacturer and data were analysed using BiaEvaluation 4.1 (Biacore AB) software. hu11E1 has a KD for 68F2 of 36 pM and a k_{off} of 4.83×10⁻⁵ s⁻¹. There is no binding to 61 H7 or to a commercial mAb binding to the same human target antigen. Therefore the binding of 11 E1 is specific to 68F2.

2.5.1.2: To Profile the Binding of the la11E1 Towards Different Antibodies in Order to Verify Specificity of la11E1 for 68F2.

In order to determine the specificity of la11 E1, an ELISA was set up. A panel of related monoclonal antibodies was coated at 2 μg/ml concentration overnight at 4° C. After coating the ELISA plate was blocked with PBS containing 1% casein for 2 hours at room temperature. The blocking solution was then replaced by 100 μl of la11E1 (llama IgG) starting with a concentration of 10 μg/ml, diluting it 3× in a dilution series until the concentration of 14 ng/ml was reached. Detection of binding took place by adding a mouse anti-llama IgG1 (27E10) monoclonal antibody and subsequently adding a donkey anti-mouse IgG antibody conjugated to HRP.

For specificity profiling the panel of antibodies tested included 68F2 and 61 H7, plus a commercial antibody binding to the same human target antigen and an irrelevant llama-derived antibody (36C4) which binds an unrelated human target antigen as isotype control. Also included was the 68F2 mismatched with an irrelevant llama-derived VH or VL. The VH68VL48 contains the VH of 68F2 but not the VL and the VH48VL68 vice versa.

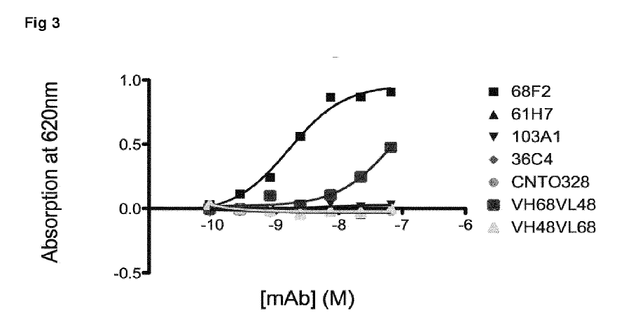

la11E1 showed high specific binding for 68F2 in this panel of antibodies (FIG. 3). Of particular importance is that the control antibody 36C4 was not recognized. This is an isotype control (same Fc variant) antibody isolated with the same technology as 68F2. There is a high chance that if 36C4 is not recognized, 68F2 could be bound very specifically in a large antibody pool (as human plasma). This was indeed confirmed by further experiments (see later).

Interestingly, the heavy chain of 68F2 is also recognized in a mismatched mAb that contains the heavy chain of 68F2 but with the light chain of an irrelevant antibody, VH68VL48. The binding is about 100-fold less for this mismatch VH68VL48 versus the full 68F2 antibody. This suggests that the main part of the binding epitope on 68F2 is localized in its variable heavy chain part but that variable light chain sequence also contributes to binding of la11E1.

2.5.1.3: To Demonstrate the Usability of la11E1 for Pharmacokinetic Studies with 129D3.

129D3 is a germlined derivative of 68F2 and only differs by few amino acids from 68F2 in the framework regions.

First la11E1 needed to demonstrate specific binding to 129D3 in the presence of blood plasma. As positive control 68F2 was used.

A Maxisorp plate was coated with 3 μg/ml la11 E1 llama format overnight at 4° C. The next day this plate was blocked with PBS containing 1% casein for 2 hours at room temperature. Meanwhile pooled day-zero plasma from three cynomolgus monkeys used were spiked with a concentration of 50 μg/ml 129D3 or 68F2. Day zero means that the monkeys were not yet treated with any type of mAb. Of this spiked pooled plasma 1 percent was added to the ELISA plate after blocking (500 ng/ml end concentration). In the adjacent wells a dilution series was made diluting a factor 2 in every well. Dilution was done in PBS containing 1 percent pooled plasma to keep the matrix in every well equal. The last well was used for 1% plasma without added monoclonal antibody to observe background signal.

68F2 or 129D3 were allowed to bind to the coating of the plate for 2 hours at room temperature. After 2 hours monkey depleted anti-human Fc polyclonal antibody conjugated to HRP (Bethyl—lot#A80-319P-14) was allowed to detect binding of the idiotype (68F2 or 129D3, which contain a human Fc part) to the anti-idiotype (la11E1 which contains a llama Fc part) for 1 hour. This was then colored with s(HS)-TMB (SDT-Reagents) and the color reaction was stopped after 10 minutes.

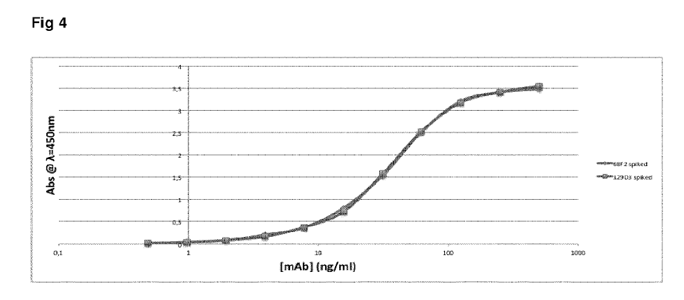

The llama IgG version of 11E1 (la11E1) was able to detect in equal fashion both 68F2 and 129D3 in a background of pooled cynomolgus monkey plasma (FIG. 4). This indicates that la11E1 can indeed be used to detect 129D3. The background signal was very low.

### 2.5.2 Characterization of 61H7 Anti-Idiotypic Antibodies

The inserts from clones 7B4, 7C6, 7C12, 8D6 and 8H7 present in the phagemid vector pCB3 were recloned into the pCB4 expression vector for larger scale Fab production. These products were used for initial characterization. Note that this is different from 11E1 where the human and llama IgG1 formats were characterized.

### 2.5.2.1: To Verify the Binding of the Selected Fabs 7B4, 7C6, 7C12, 8D6 and 8H7 Against its Target 61 H7 Assess the Specificity of Binding.

96 well plates were coated with human 61H7 target antibody. Purified Fabs of 7B4, 7C6, 7C12, 8D6 and 8H7 were added to the wells in a 3 fold dilution series (800 nM, 267 nM, 89 nM, 30 nM, 10 nM and 3.3 nM). Detection was performed with anti-MYC HRP. This experiment was performed twice on two different days. In the first experiment following negative controls were included: commercial antibody binding the same human target antigen, 68F2 and 36C4 (an isotype control llama-derived antibody against an unrelated human target antigen). The negative control molecules were coated on the plate and incubated with 200 nM of the Fabs. In the second experiment the negative controls were the commercial antibody against the same human target protein, Llama 68F2 and human 68F2. In addition, llama 61H7 and 103A1 (a minor sequence variant of 61H7) were included to verify that these versions of 61H7 were also recognized by the Fabs.

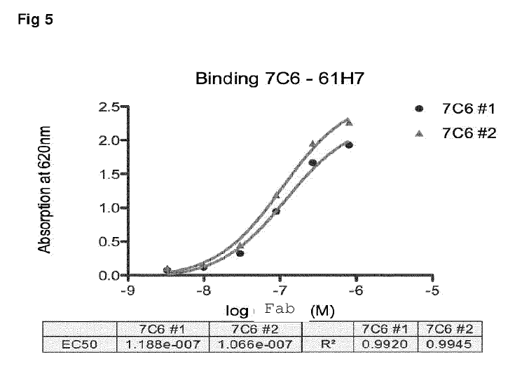

The results of this assay demonstrated that:
7C6 Fab clearly binds specifically to its target 61H7
7C6 Fab binds to the idiotype mAb human 61H7 with an approximate EC50 of 1.13×10⁻⁷ M (FIG. 5)
7C6 does not bind the negative controls human 68F2, llama 68F2, commercial antibody against the same target and 36C4
Llama 61H7 and llama 103A1 (similar to 61H7) is recognized by 7C6

### Example 3

### Anti-Idiotype Antibodies to Llama Fabs Binding a Human TNF-Ligand Family Antigen

### 3.1 Target Antigen

The target antigen for this example was a llama-derived monoclonal antibody which specifically binds to a human target protein which is a member of the TNF-ligand family. This monoclonal antibody consists of llama-derived variable Vlambda region (denoted 41D12) linked to Clambda region and the llama-derived VH to CH1-hinge-CH2CH3 of human IgG1.

Fab 41D12 is a germlined variant of a llama-originating Fab denoted 27B3. The llama-derived Fab 27B was itself raised by immunisation of a fully outbred llama with cells expressing the target antigen (human TNF-ligand family member). Fab 41D12 is a variant derived from 27B3 by the introduction of 15 amino acid substitutions within the framework regions, in order to increase the overall sequence identity up to 95.7% with the closest human germlines. 41D12 and 27B3 exhibit the same binding specificity and affinity for the target human antigen, however Fab 41D12 is more suitable for use as a human therapeutic agent, particularly when formatted as a monoclonal antibody with a fully human Fc region.

There is a commercial interest in developing anti-idiotype Fabs (or mAbs) which bind specifically to the Fab 41D12 (i.e. the germlined variant of 27B3), particularly to facilitate pharmacokinetic (PK) studies required during clinical trials of mAbs containing Fab 41D12.

### 3.4 Immunization of Llamas

Two llamas were immunized with 27B3-llama-Fc. 27B3 is the non-germlined version of 41D12, i.e. the framework regions in both the VH and VL domains have “fully llama” sequence. The use of the llama-originating Fab 27B3, rather than the germlined variant 41D12, for immunisation of llamas ensures that the immune response of the llama is directed against the antigen-binding regions of the Fab (anti-idiotype) rather than any epitopes created by the amino acid substitutions introduced in the framework regions of the germlined variant 41D12. Similarly, the llama-Fc was used for immunization to avoid an immune response against the Fc part.

To assess the strength of the immune response generated against 41D12, pre-immune and immune sera from the llamas were tested for the presence of 41D12 specific antibodies on directly coated 41D12 in an ELISA. Detection was done using an anti-llama IgG1. The results obtained (not shown) demonstrated a good immune response from one of the immunized llamas and a weaker response from the second llama.

### 3.5 Library Construction

PBLs isolated from the immunized llamas were used for RNA extraction, RT-PCR and cloning of Fab-encoding gene segment in a phagemid (pCB3) using the strategy described by De Haard H, et al., J. Biol. Chem. 274, 1999 with the primers described in WO 2010/001251, to obtain large libraries.

Independent VλCλ and VκCκ libraries were constructed using a two step PCR, in which 25 cycles with non tagged primers was done followed by 10 cycles with the tagged version of these primers. The VHCH1 libraries were built in parallel using the same approach. The sublibraries were made in pCB3. The sizes of these libraries were between 10⁸ and 10⁹ cfu.

Next, the light chain from the VλCλ and VκCκ libraries were re-cloned separately in the VHCH1-containing vector to create the “Lambda” and “Kappa” llama Fab-library respectively. Quality control of the libraries was routinely performed using PCR. The sizes of these libraries were between 10⁹ and 10¹⁰ cfu.

### 3.6 Selection and Screening

Phage display was used to recruit a diverse panel of llama Fabs binding to 41D12. Selections were done on directly coated 41D12, and counter selection was done by adding 20% human serum.

For each selection, 24 individual clones were grown in a 96-deep well plate (1 ml expressions) and periplasmic fractions were prepared and tested in a binding ELISA. Binding was measured on 27B3-llama-Fc, on 41D12 and on a negative control mAb denoted 57A10 (mAb containing llama-derived Fabs which bind to a different, unrelated human protein). Detection was done using an anti-c-myc-HRP mAb.

The results (not shown) demonstrated that in all the libraries Fabs are found that specifically bind to 41D12 and not to 57A10.

Periplasmic extracts were tested for off rate in Biacore on a 41D12 and 57A10 coated CM5 chip. The results for some of the clones are shown in Table 3. High affinity and specific binding to the idiotypic antibody 41D12 could be demonstrated.

TABLE 3
Binding of periplasmic fractions containing llama-
derived anti-idiotypic Fabs to the idiotypic mAb
and negative control mAb, measured by Biacore

| Clone | Ro for 41D12 [RU] | Ro for 57A10 [RU] | Off rate [s−1] |
|---|---|---|---|
| 66B2 | 128 | 2 | 1.60E−03 |
| 66E1 | 148 | 2 | 1.63E−03 |
| 66D1 | 133 | 7 | 4.46E−03 |
| 66B1 | 33 | 13 | 2.76E−03 |
| 66D3 | 282 | 8 | 1.41E−03 |
| 66E3 | 266 | 1 | 2.64E−04 |
| 66C8 | 12 | 3 | 7.90E−04 |
| 66B3 | 238 | 5 | 1.45E−03 |
| 67H3 | 25 | 7 | 1.20E−03 |
| 66E8 | 44 | 6 | 4.84E−03 |
| 66H1 | 56 | 8 | 1.54E−03 |

3.7 Binding of Llama-Derived Anti-Idiotypic mAbs to Idiotypic mAb Spiked in Human Plasma by ELISA

The aim of this ELISA was to determine if 41D12 can be specifically detected in human plasma using the anti-idiotypic mAbs, demonstrating the utility of the anti-idiotypic mAbs as tools for development of human PK assays.

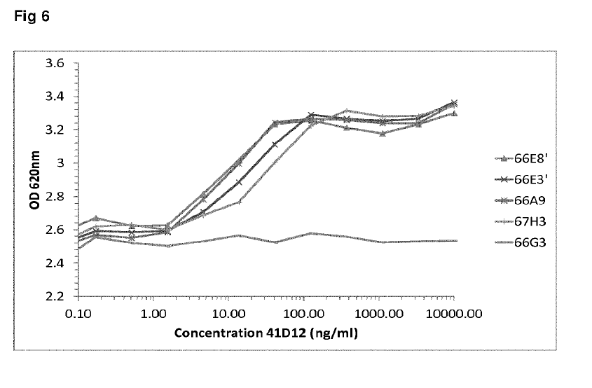

Anti-idiotypic mAbs 66E8, 66E3, 66A9 and 67H3 and an irrelevant mAb (66G3 also with llama Fc) were coated in a Nunc 96-well microtiter plate at 5 μg/ml overnight at 4° C. After washing, the plate was blocked for 2 hours with 2% BSA then 41D12 was applied at a serial dilution starting at 10 μg/ml, making 3 fold dilutions in 1% human plasma. The samples were incubated for 2 hours at RT. After washing, 41D12 was detected using Anti-human Fc HRP (Jackson 109-035-008) at a 5000 fold dilution. Plates were washed and TMB substrate was used for staining, OD was measured at OD620 nm. Results are shown in FIG. 6.

The results demonstrate that 41D12 can be detected at high sensitivity in human plasma (detection limit around 5 ng/ml). The background in this assay is however very high. This is because the Anti-human Fc HRP (Jackson 109-035-008) also detects the llama-Fc of the coated mAbs. In this respect the previously used detection antibody (monkey IgG depleted anti-human Fc polyclonal antibody conjugated to HRP from Bethyl) is preferred, because it does not cross-react with llama IgG (see example 2.5.1.3).

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. Moreover, all aspects and embodiments of the invention described herein are considered to be broadly applicable and combinable with any and all other consistent embodiments, including those taken from other aspects of the invention (including in isolation) as appropriate.

Various publications are cited herein, the disclosures of which are incorporated by reference in their entireties.

1-17. (canceled)

18. A method for preparing a recombinant antigen binding polypeptide that specifically binds to a target antigen, said antigen binding polypeptide comprising a VH domain and a VL domain, wherein at least one hypervariable loop or complementarity determining region (CDR) in the VH domain or the VL domain is obtained from a species in the family Camelidae, wherein the target antigen is a polypeptide antigen that exhibits at least 90% amino acid sequence identity with a protein from said species in the family Camelidae over a sequence comparison window, said method comprising the step of:
(a) immunizing a species in the family Camelidae with the target antigen, thereby raising a conventional antibody to said target antigen.

19. (canceled)

20. (canceled)

21. The method of claim 18, wherein the species in the family Camelidae is selected from the group consisting of camel, llama, dromedary, vicuna, guanaco, and alpaca.

22-33. (canceled)

34. The method of claim 18, wherein the sequence comparison window includes at least the epitope for the antigen binding polypeptide.

35. The method of claim 18, wherein the sequence comparison window comprises at least one domain of the target antigen, including the epitope for the antigen binding polypeptide.

36. The method of claim 18, wherein the sequence comparison window includes the full length of the target antigen.

37. The method of claim 18, further comprising the steps of:
(b) isolating Camelidae nucleic acid encoding at least one hypervariable loop or complementarity determining region (CDR) of the VH and/or the VL domain of a Camelidae conventional antibody immunoreactive with said target antigen;
(c) preparing a polynucleotide comprising a nucleotide sequence encoding hypervariable loop(s) or complementarity determining region(s) having amino acid sequence identical to the hypervariable loop(s) or complementarity determining region(s) encoded by the nucleic acid isolated in step (a), which polynucleotide encodes an antigen binding polypeptide comprising a VH domain and a VL domain that specifically binds to said target antigen; and
(d) expressing said antigen binding polypeptide from the recombinant polynucleotide of step (c), wherein said antigen binding polypeptide is not identical to the Camelidae conventional antibody of step (b).

38. The method of claim 37, wherein step (b) comprises isolating Camelidae nucleic acid encoding the VH domain and/or the VL domain of said antibody and altering the sequence of said nucleic acid such that it encodes a VH domain and/or a VL domain with one or more amino acid substitutions, deletions, or additions.

39. The method of claim 38, wherein the recombinant polynucleotide prepared in step (c) additionally comprises a nucleotide sequence encoding one or more constant domains of a human antibody.

40. The method of claim 37, wherein the recombinant polynucleotide prepared in step (c) additionally comprises a nucleotide sequence encoding one or more constant domains of a human antibody.

41. The method of claim 37, wherein the species in the family Camelidae is selected from the group consisting of camel, llama, dromedary, vicuña, guanaco, and alpaca.

42. The method of claim 37, wherein the sequence comparison window includes at least the epitope for the antigen binding polypeptide.

43. The method of claim 37, wherein the sequence comparison window comprises at least one domain of the target antigen, including the epitope for the antigen binding polypeptide.

44. The method of claim 37, wherein the sequence comparison window includes the full length of the target antigen.

45. The method of claim 37, wherein the target antigen is a highly conserved antigen.

46. The method of claim 45, wherein the highly conserved antigen exhibits at least 95% amino acid sequence identity with the homologous protein from the species in the family Camelidae over the sequence comparison window.

47. The method of claim 45, wherein the highly conserved antigen is a polypeptide from a species selected from the group consisting of human, non-human primate, mouse, rat, pig, dog, guinea pig, rabbit, sheep, cow, and chicken.

48. The method of claim 45, wherein the highly conserved antigen is a human polypeptide.

49. The method of claim 37, wherein the target antigen is a self-antigen from the species in the family Camelidae.

50. The method of claim 37, wherein the target antigen is a camelid-derived antigen that exhibits at least 90% amino acid sequence identity with a self-antigen from the species in the family Camelidae over the sequence comparison window.

51. The method of claim 37, wherein the target antigen is the variable region of an antibody obtained from the species in the family Camelidae.

52. The method of claim 37, wherein the target antigen is a variant of a variable region of an antibody obtained from the species in the family Camelidae which exhibits at least 90% amino acid sequence identity with the variable region of the antibody obtained from said species in the family Camelidae over a sequence comparison window that includes the entire variable region of said antibody.
